(19) United States
(12) Patent Application Publication
(10) Pub. No.: US 2023/0358706 A1
(43) Pub. Date: Nov. 9, 2023

(54) DEVICES AND METHODS FOR URINE SAMPLE ANALYSIS

(71) Applicant: Roche Sequencing Solutions, Inc., Pleasanton, CA (US)

(72) Inventors: Keynttisha JEFFERSON, Pleasanton, CA (US); Pantea GHEIBI, Pleasanton, CA (US)

(21) Appl. No.: 18/246,009

(22) PCT Filed: Apr. 15, 2021
(86) PCT No.: PCT/EP2021/059784

§ 371 Date: Mar. 20, 2023

Related U.S. Application Data
(60) Provisional application No. 63/011,355, filed on Apr. 17, 2020.

(51) Int. Cl. G01N 27/447; G01N 27/327

(52) U.S. Cl. CPC G01N 27/4473; G01N 27/3271

(57) ABSTRACT

The present disclosure generally relates to devices and methods for effecting epitachophoresis in order to isolate/purify analytes from urine samples or other samples comprising high salt concentrations, e.g., sodium or potassium salts. Epitachophoresis may be used to effect sample analysis, such as by selective separation, detection, extraction, and/or pre-concentration of target analytes such as, for example, DNA, RNA, and/or other biological molecules. Said target analytes may be collected following epitachophoresis and used for desired downstream applications and further analysis.

### FIELD OF THE ART

The present disclosure generally relates to the field of electrophoresis for sample analysis, and more particularly relates to urine sample analysis or the analysis of other high salt comprising biological samples by selective separation, detection, extraction, isolation, purification, and/or (pre-) concentration of samples such as, for example, urine samples comprising nucleic acids, through devices and methods for epitachophoresis.

### BACKGROUND

The concept of a “liquid biopsy” has gained traction in recent years. Instead of taking a sample from solid tissue, liquid biopsies capture biomarkers such as cells, extracellular vesicles such as exosomes, and/or cell-free molecules such as DNA, RNA, or proteins. These biomarkers can be collected in biofluids such as blood (or plasma or serum), urine, sputum, and the like. The presence of these biomarkers can be associated with, for example, cancers, tumors, autoimmune diseases, cardiovascular events, viruses, bacterial or pathogenic infection, or related to a drug response. These molecules are often associated with extracellular bodies such as exosomes or may be “cell-free” in the fluid.

Liquid biopsies can be conducted using any of several biofluids, and can be minimally invasive (e.g., blood collection by phlebotomy) or non-invasive (urine collection). Liquid biopsies are thus attractive for ease of collection, ease of repeat collection for patient monitoring, higher likelihood of patient acquiescence, familiarity of sample collection to patients, and less specialized collection sites.

One drawback of liquid biopsies is that the concentration of biomarker, e.g., nucleic acid, can be relatively low, so that a large volume is needed to obtain sufficient material for downstream analysis. In the case of nucleic acids, conventional techniques using nucleic acid capture kits and devices are generally designed for small sample volumes, which are typically in the range of 0.2-1 mL. As such, in spite of being a biomarker-rich resource, efficient isolation and purification of biomarkers from urine has thus far proven challenging. Moreover, conventional techniques for the extraction, isolation and/or purification of biomarkers from urine are often time-consuming, yield low quality product, and are not well suited to automation. Therefore, further development of devices and methods for analyzing urine samples, such as for detection of one or more biomarkers, is needed.

### BRIEF SUMMARY

A system may concentrate the urine sample to form a concentrated urine sample. The concentrated urine sample may have a concentration of the one or more target analytes that is at least 10 times higher than an initial concentration of the one or more target analytes in the urine sample. The system may add the concentrated urine sample to a first electrolyte to form a first mixture. The system may apply a voltage difference between a first electrode and a second electrode. The first electrode is disposed in the first mixture. The second electrode is disposed in a second electrolyte. The first electrolyte may be different from the second electrolyte. The system may flow, using the voltage difference, the one or more target analytes in one or more focused zones to the second electrode. The system may collect the one or more target analytes by collecting a second mixture comprising the one or more focused zones. The concentration of any of the one or more target analytes in the second mixture is higher than the concentration of the respective target analyte in the concentrated urine sample. No further extraction of the target analytes may be needed before further analysis.

In embodiments, a system may include an epitachophoresis (“ETP”) device. The device may include a circular first electrode disposed at an outer edge of a circular channel. The device may also include a sample collection reservoir in the center of the circular channel. The system may further include a second electrode. The second electrode is configured to be in closer electrical communication with the sample collection reservoir than the circular first electrode is with the sample collection reservoir. Additionally, the device may include a power supply configured to deliver a voltage difference between the circular first electrode and the second electrode. The system may also include a sample concentrating device configured to increase the concentration of one or more target analytes within a sample at least 10 times higher.

The present disclosure generally relates to a method of isolating and/or purifying one or more target analytes from a urine or other high salt comprising biological sample, such as sodium or potassium salts, which sample potentially comprises one or more target analytes, wherein said method comprises: a. providing a device for effecting epitachophoresis; b. providing the sample comprising said one or more target analytes; c. performing one or more epitachophoresis runs by effecting ETP using said device to focus said one or more target analytes into one or more focused zones; and d. collecting said one or more target analytes by collecting said one or more focused zones comprising said one or more target analytes; thereby obtaining one or more isolated and/or purified target analytes, optionally wherein said target analytes comprise one or more nucleic acids. In some embodiments, prior to step c., a sample solution may be prepared by effecting one or more sample pretreatment steps on said urine or other high salt comprising biological sample. In some embodiments, the sample may comprise a urine sample. In some embodiments, the sample pretreatment may comprise one or more of vacuum filtration, desalting, buffer exchange, extracellular vesicle enrichment, exosome enrichment, cell lysis, protein degradation, centrifugation-based cell-removal, and/or concentration steps. In some embodiments, said desalting, buffer exchange, extracellular vesicle enrichment, exosome enrichment step, and/or concentration steps may be centrifugation-based.

In some embodiments, the one or more target analytes may comprise any one or more of one or more nucleic acids; one or more proteins; one or more cells; one or more extracellular vesicles; one or more one or more exosomes, microvesicles, and/or apoptotic bodies, optionally one or more urinary exosomes; and/or one or more biomarkers. In some embodiments, the one or more target analytes may comprise DNA and/or RNA. In some embodiments, the one or more target analytes may comprise one or more circulating nucleic acids. In some embodiments, the isolated and/or purified target analytes may comprise DNA and/or RNA. In some embodiments, the quantity of nucleic acids isolated and/or purified may be greater as compared to the quantity of nucleic acids obtained using a column-based or bead-based protocol as measured by a fluorometer-based method. In some embodiments, the quality of nucleic acids isolated and/or purified may be higher as compared to the quality of nucleic acids obtained using a column-based or bead-based protocol as measured by a quality control qPCR-based method. In some embodiments, 1.25 times or more, 1.5 times or more, 1.75 times or more, 2.0 times or more, 2.25 times or more, 2.5 times or more, 2.75 times or more, 3 time or more, 4 times or more, 5 times or more, 10 times or more, 100 times or more, or 1000 times or more nucleic acids may be collected as compared to the quantity of nucleic acids obtained using a column-based or bead-based protocol. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of the one or more nucleic acids comprised in the original sample being isolated and/or purified and collected. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of the isolated and/or purified one or more nucleic acids such as measured by an analytical technique to determine the composition of isolated/purified sample comprising one or more nucleic acids. In some embodiments, the quality of said isolated and/or purified nucleic acids may be determined by quality control qPCR. In some embodiments, the isolated and/or purified nucleic acids may be of any desired size. In some embodiments, the isolated and/or purified nucleic acids may be 5 nt or less, 10 nt or less, 20 nt or less, 30 nt or less, 50 nt or less, 100 nt or less, 1000 nt or less, 10,000 nt or less, 100,000 nt or less, 1,000,000 nt or less, or 1,000,000 nt or more in size. In some embodiments, the sample volume may be 0.25 mL or less, 0.25 mL or more, 0.5 mL or more, 0.75 mL or more, 1.0 mL or more, 2.5 mL or more, 5.0 mL or more, 7.5 mL or more, 10.0 mL or more, 12.5 mL or more, or 15.0 mL or more, 20.0 mL or more, 25.0 mL or more, 30.0 mL or more, 40.0 mL or more, or 50 mL or more. In some embodiments, the sample volume may be from about 1 mL to about 50 mL.

Moreover, the present disclosure generally relates to a method of isolating and/or purifying one or target analytes, optionally one or more nucleic acids, from a urine sample, wherein said method comprises: a. providing a device for effecting epitachophoresis (ETP); b. providing a urine sample comprising one or more target analytes; c. preparing a urine sample solution by effecting one or more pre-treatment steps on said urine sample; d. performing one or more epitachophoresis runs by effecting ETP using said device to focus said one or more target analytes into one or more focused zones, e.g., as one or more ETP bands; and e. collecting said one or more target analytes by collecting said one or more focused zones comprising said one or more target analyte; thereby obtaining one or more isolated and/or purified target analytes, optionally wherein said target analytes comprise any one or more of nucleic acids, cell-free nucleic acids, circulating tumor nucleic acids, biomarkers, proteins, and/or extracellular vesicles.

Furthermore, the present disclosure generally relates to a method of isolating and/or purifying one or target analytes, optionally one or more nucleic acids, from a urine sample, wherein said method comprises: a. providing a device for effecting epitachophoresis (ETP); b. providing a urine sample comprising one or more target analytes; c. preparing a urine sample solution by effecting one or more pre-treatment steps on said urine sample, wherein said pre-treatment steps comprise one or more of one or more vacuum filtration, desalting, buffer exchange, extracellular vesicle enrichment, exosome enrichment, cell lysis, protein degradation, centrifugation-based cell-removal and/or concentration steps; d. performing one or more epitachophoresis runs by effecting ETP using said device to focus said one or more target analytes into one or more focused zones, e.g., as one or more ETP bands; and e. collecting said one or more target analytes by collecting said one or more focused zones comprising said one or more target analyte; thereby obtaining one or more isolated and/or purified target analytes, optionally wherein said target analytes comprise any one or more of nucleic acids, cell-free nucleic acids, circulating tumor nucleic acids, biomarkers, proteins, and/or extracellular vesicles.

Moreover, the present disclosure generally relates to a method of isolating and/or purifying one or nucleic acids, optionally one or more circulating nucleic acids, from a urine sample, wherein said method comprises: a. providing a device for effecting epitachophoresis (ETP); b. providing a urine sample comprising one or more target analytes; c. preparing a urine sample solution by effecting one or more pre-treatment steps on said urine sample, wherein said pre-treatment steps comprise one or more of one or more vacuum filtration, desalting, buffer exchange, extracellular vesicle enrichment, exosome enrichment, cell lysis, protein degradation, centrifugation-based cell-removal and/or concentration steps; d. performing one or more epitachophoresis runs by effecting ETP using said device to focus said one or more target analytes into one or more focused zones, e.g., as one or more ETP bands; and e. collecting said one or more target analytes by collecting said one or more focused zones comprising said one or more target analyte; thereby obtaining one or more isolated and/or purified nucleic acids.

In some embodiments of the method described herein, said isolated and/or purified one or more target analytes are subjected to one or more in vitro diagnostic (“IVD”) assays.

### BRIEF DESCRIPTION OF THE DRAWINGS

FIG. 1 provides a schematic representation of an exemplary device for effecting epitachophoresis.

FIG. 2A provides a schematic representation of a top view of an exemplary device for effecting epitachophoresis. In FIG. 2A, numbers 1-7 refer to the following: 1. Outer circular electrode; 2. Terminating electrolyte reservoir; 3. Leading electrolyte, optionally contained within a gel or otherwise hydrodynamically separated from the terminating electrolyte; 4. Leading electrolyte electrode/collection reservoir; 5. Central electrode; 6. Electric power supply; and 7. Boundary between leading and terminating electrolytes with sample ions focused in between; and the symbols r and d are used to represent the leading electrolyte reservoir radius and distance migrated by the LE/TE boundary, respectively.

FIG. 2B provides a schematic representation of a side view of an exemplary device for effecting epitachophoresis. In FIG. 2B, numbers 1-8 refer to the following: 1. Outer circular electrode; 2. Terminating electrolyte reservoir; 3. Leading electrolyte, optionally contained within a gel or otherwise hydrodynamically separated from the terminating electrolyte; 4. Leading electrolyte electrode/collection reservoir; 5. Center electrode; 6. Electric power supply; 7. Boundary between leading and terminating electrolytes with sample ions focused in between; and 8. Bottom support; and the symbols r and d are used to represent the leading electrolyte reservoir radius and distance migrated by the LE/TE boundary, respectively.

FIG. 3 provides a schematic representation of an exemplary device for effecting epitachophoresis.

FIG. 4 provides a schematic representation of an exemplary device for effecting epitachophoresis. In FIG. 4, the numbers 1-10 refer to the following: 1. Outer circular electrode; 2. Terminating electrolyte reservoir; 3. Leading electrolyte, optionally contained within a gel or otherwise hydrodynamically separated from the terminating electrolyte; 4. Opening to leading electrolyte/collection reservoir; 5. Center electrode; 6. Electric power supply; 7. Boundary between leading and terminating electrolytes with sample ions focused in between; 8. Bottom support; 9. Tube connecting device to a leading electrolyte reservoir; 10. Leading electrolyte reservoir.

FIG. 5 provides a schematic representation of an exemplary device for effecting epitachophoresis wherein the sample is loaded in between loading the leading and terminating electrolytes.

FIG. 6A provides a schematic representation of a device for effecting epitachophoresis.

FIG. 6B provides a graph representing the travelled distance d in cm vs. the relative velocity at the distance d when an exemplary device for epitachophoresis (FIG. 6A) is operated using constant current. For the example presented in FIG. 6B, a radius value of 5 and starting velocity value of 1 were used.

FIG. 7 provides an image of an epitachophoresis device that was used to concentrate a sample.

FIG. 8A provides an image of an exemplary device for epitachophoresis.

FIG. 8B provides an image of an exemplary device for epitachophoresis that was used to focus a sample into a focused zone.

FIG. 8C provides an image of an exemplary device for epitachophoresis that was used to focus a sample into a focused zone.

FIG. 9A provides an image of an exemplary device for epitachophoresis.

FIG. 9B provides a schematic representation of an exemplary device for epitachophoresis that was used. In FIG. 9B, the numbers refer to dimensions in millimeters.

FIG. 9C provides an image of an exemplary device for epitachophoresis that was used to focus a sample into a focused zone.

FIG. 9D provides an image of an exemplary device for epitachophoresis that was used to focus a sample into a focused zone.

FIG. 10 provides an image of an exemplary device for epitachophoresis that was used to focus a sample into a focused zone.

FIG. 11 provides an image of an exemplary device for epitachophoresis that was used to separate and to focus two different samples into focused zones.

FIG. 12 provides an image of an exemplary device for epitachophoresis.

FIG. 13A provides an image of an exemplary epitachophoresis device.

FIG. 13B provides a schematic of an exemplary epitachophoresis device. “a” corresponds to the central collection well and “b” corresponds to the leading electrolyte reservoir.

FIG. 14A provides an image of an exemplary conductivity measurement probe for use in an epitachophoresis device.

FIG. 14B provides an image showing a closer view of the conductivity measurement probe shown in FIG. 14A.

FIG. 15A provides an image of an exemplary epitachophoresis device with a conductivity probe.

FIG. 15B provides a conductivity trace for a run of an exemplary epitachophoresis device.

FIG. 16A provides images of an exemplary epitachophoresis device with conductivity detecting probes placed underneath the semipermeable membrane.

FIG. 16B provides images of an exemplary bottom substrate incorporating two conductivity detecting probes connected through dedicated channels within the central pillar.

FIG. 17A provides an image of an exemplary epitachophoresis device, demonstrating the focusing of a fluorescein-labeled DNA ladder sample.

FIG. 17B provides a trace showing the resistivity change of the LE/TE transition monitored by a surface conductivity cell for a run of an exemplary epitachophoresis device.

FIG. 17C provides absorbance spectra of the original sample and the collected fraction for the DNA ladder sample before and after an epitachophoresis run.

FIG. 17D provides electropherograms for the DNA ladder sample before and after an epitachophoresis run, as measured via Bioanalyzer separations.

FIG. 18 provides voltage profiles for three independent ETP runs.

FIG. 19A provides an image of an ETP device during an ETP run.

FIG. 19B provides a fluorescence-based image of an ETP device taken during an ETP run.

FIG. 19C provides an image of an ETP device taken during an ETP run.

FIG. 19D provides an electropherogram for the focused and collected ETP sample analyzed.

FIG. 20A provides images captured using an infrared-based thermal imaging camera during at ETP run.

FIG. 20B provides a fluorescence-based image captured during an ETP run.

FIG. 20C presents data related to the voltage change and temperature change over time during an ETP run.

FIG. 21 provides an image of an ETP device, and accessories. a. denotes the ETP device; b denotes the rectangular lid of the ETP device; c. denotes the circular lid of the ETP device; and d. denotes the Teflon stick used to adjust the position of the mobile central piston of the ETP device.

FIG. 22 provides an image of the ETP experimental setup.

FIG. 23 provides time lapse images of isolation/purification of cfDNA from 1 mL of plasma.

FIG. 24 provides (QUBIT-based) measurements of the concentration of cfDNA isolated/purified by ETP-based isolation/purification followed by a bead-based cleanup. Data from measurements of the concentration of cfDNA isolated/purified by spin-column or by a bead-based method are also presented.

FIG. 25 provides data from size-based analysis of cfDNA and DNA ladder isolated/purified from a plasma sample spiked with DNA ladder by ETP-based isolation/purification.

FIG. 26 provides data from size-based analysis of the cfDNA isolated/purified from a plasma sample by ETP-based isolation/purification.

FIG. 27 provides data from size-based analysis of the cfDNA isolated/purified by ETP-based isolation/purification.

FIG. 28 provides measurements of the concentration of ctDNA isolated/purified by ETP-based isolation/purification followed by a bead-based cleanup. Data are also presented for measurements of the concentration of ctDNA isolated/purified by a spin column-based method.

FIG. 29 provides data from electrophoretic-based analysis of the ETP upper markers generated by digesting a vector with three restriction enzymes.

FIG. 30 shows a process for isolating and/or purifying one or more target analytes from a urine sample according to embodiments of the present invention.

FIG. 31 shows a system for isolating and/or purifying one or more target analytes from a urine sample according to embodiments of the present invention.

FIG. 32 showns examples of subsystems that may be used in any of the computer systems mentioned herein.

### TERMS

As used herein the singular forms “a”, “an”, and “the” include plural referents unless the context clearly dictates otherwise. All technical and scientific terms used herein have the same meaning as commonly understood to one of ordinary skill in the art to which this invention belongs unless clearly indicated otherwise.

As used herein, the term “isotachophoresis” generally refers to the separation of charged particles by using an electric field to create boundaries or interfaces between materials (e.g., between the charged particles and other materials in a solution). ITP generally uses multiple electrolytes, where the electrophoretic mobilities of sample ions are less than that of a leading electrolyte (LE) and greater than that of a trailing electrolyte (TE) that are placed in a device for ITP. The leading electrolyte (LE) generally contains a relatively high mobility ion, and a trailing electrolyte (TE) generally contains a relatively low mobility ion. The TE and LE ions are chosen to have effective mobilities respectively lower and higher than target analyte ions of interest. That is, the effective mobility of analyte ions is higher than that of the TE and lower than that of the LE. These target analytes have the same sign of charge as the LE and TE ions (i.e., a co-ion). An applied electric field causes LE ions to move away from TE ions and TE ions to trail behind. A moving interface forms between the adjacent and contiguous TE and LE zones. This creates a region of electric field gradient (typically from the low electric field of the LE to the high electric field of the TE). Analyte ions in the TE overtake TE ions but cannot overtake LE ions and accumulate (“focus” or form a “focused zone”) at the interface between TE and LE. Alternately, target ions in the LE are overtaken by the LE ions; and also accumulate at interface. With judicious choice of LE and TE chemistry, ITP is fairly generally applicable, can be accomplished with samples initially dissolved in either or both the TE and LE electrolytes, and may not require very low electrical conductivity background electrolytes.

As used herein, the term “epitachophoresis” generally refers to methods of electrophoretic separation that are performed using a circular or spheroid and/or concentric device and/or circular and/or concentric electrode arrangement, such as by use of the circular/concentric and/or polygonal devices as described herein. Due to a circular/concentric or another polygonal arrangement that is used during epitachophoresis; unlike conventional isotachophoresis devices, the cross section area changes during migration of ions and zones, and the velocity of the zone movement is not constant in time due to the changing cross sectional area. Thus, an epitachophoretic arrangement does not strictly follow conventional isotachophoretic principles, wherein the zones migrate with constant velocities. Notwithstanding these significant differences as shown herein epitachophoresis can be used to efficiently separate and focus charged particles by using an electric field to create boundaries or interfaces between materials that may have different electrophoretic mobilities (e.g., between the charged particles and other materials in a solution). LE and TE, as described for use with ITP, can be used for epitachophoresis as well. In some embodiments, epitachophoresis may be effected using constant current, constant voltage, and/or constant power. In some embodiments, epitachophoresis may be effected using varying current, varying voltage, and/or varying power. In some embodiments, epitachophoresis may be effected within the context of devices and/or an arrangement of electrodes whose shape may be described in general as circular or spheroid, such that the basic principles of epitachophoresis may be accomplished as described herein. In some embodiments, epitachophoresis may be effected within the context of devices and/or an arrangement of electrodes whose shape may be described in general as polygons, such that the basic principles of epitachophoresis may be accomplished as described herein. In some embodiments, epitachophoresis may be effected by any non-linear, contiguous arrangement of electrodes, such as electrodes arranged in the shape of a circle and/or electrodes arranged in the shape of a polygon.

As used herein, the terms “in vitro diagnostic application (IVD application)”, “in vitro diagnostic method (IVD method)”, “in vitro diagnostic assay”, and the like, generally refer to any application and/or method and/or device that may evaluate a sample for a diagnostic and/or monitoring purposes, such as identifying a disease in a subject, optionally a human subject. In some embodiments, said sample may comprise nucleic acids and/or target nucleic acids from a subject and/or from a sample, optionally further wherein said nucleic acids originated from a urine sample. In some embodiments, an epitachophoresis device may be used as an in vitro diagnostic device. In some embodiments, a target analyte that has been concentrated/enriched/isolated/purified through epitachophoresis may be used in a downstream in vitro diagnostic assay. In some embodiments, an in vitro diagnostic assay may comprise nucleic acid sequencing, e.g., DNA sequencing, e.g., RNA sequencing. In some embodiments, and IVD assay may comprise gene expression profiling. In some embodiments, an in vitro diagnostic method may be, but is not limited to being, any one or more of the following: staining, immunohistochemical staining, flow cytometry, FACS, fluorescence-activated droplet sorting, image analysis, hybridization, DASH, molecular beacons, primer extension, microarrays, CISH, FISH, fiber FISH, quantitative FISH, flow FISH, comparative genomic hybridization, blotting, Western blotting, Southern blotting, Eastern blotting, Far-Western blotting, Southwestern blotting, Northwestern blotting, and Northern blotting, enzymatic assays, ELISA, ligand binding assays, immunoprecipitation, ChIP, ChIP-seq, ChIP-ChiP, radioimmunoassays, fluorescence polarization, FRET, surface plasmon resonance, filter binding assays, affinity chromatography, immunocytochemistry, gene expression profiling, DNA profiling with PCR, DNA microarrays, serial analysis of gene expression, real-time polymerase chain reaction, differential display PCR, RNA-seq, mass spectrometry, DNA methylation detection, acoustic energy, lipidomic-based analyses, quantification of immune cells, detection of cancer-associated markers, affinity purification of specific cell types, DNA sequencing, next-generation sequencing, detection of cancer-associated fusion proteins, and detection of chemotherapy resistance-associated markers.

As used herein, the terms “leading electrolyte” and “leading ion” generally refer to ions having a higher effective electrophoretic mobility as compared to that of the sample ion of interest and/or the trailing electrolyte as used during ITP and/or epitachophoresis. In some embodiments, leading electrolytes for use with cationic epitachophoresis may include, but are not limited to including, chloride, sulphate and/or formate, buffered to desired pH with a suitable base, such as, for example, histidine, TRIS, creatinine, and the like. In some embodiments, leading electrolytes for use with anionic epitachophoresis may include, but are not limited to including, potassium, ammonium and/or sodium with acetate or formate. In some embodiments, an increase of the concentration of the leading electrolyte may result in a proportional increase of the sample zone and a corresponding increase in electric current (power) for a given applied voltage. Typical concentrations generally may be in the 10-100 mM range; however, higher concentrations may also be used.

As used herein, the terms “trailing electrolyte”, “trailing ion”, “terminating electrolyte”, and “terminating ion” generally refer to ions having a lower effective electrophoretic mobility as compared to that of the sample ion of interest and/or the leading electrolyte as used during ITP and/or epitachophoresis. In some embodiments, trailing electrolytes for use with cationic epitachophoresis may include, but are not limited to including, IVIES, MOPS, acetate, glutamate and other anions of weak acids and low mobility anions. In some embodiments, trailing electrolytes for use with anionic epitachophoresis may include, but are not limited to including, reaction hydroxonium ion at the moving boundary as formed by any weak acid during epitachophoresis.

As used herein, the term “focused zone(s)” generally refers to a volume of solution that comprises a component that has been concentrated (“focused”) as a result of performing epitachophoresis. A focused zone may be collected or removed from a device, and said focused zone may comprise an enriched and/or concentrated amount of a desired sample, e.g., a target analyte, e.g., a target nucleic acid. In the epitachophoresis methods described herein the target analyte generally becomes focused in the center of the device, e.g., a circular or spheroid or other polygonal shaped device.

As used herein, the terms “band” and “ETP band” generally refer to a zone (e.g. focused zone) of ion, analyte, or sample that travels separately from other ions, analytes, or samples during electrophoretic (e.g., isotachophoretic, or epitachophoretic) migration. A focused zone within an epitachophoresis device may alternatively be referred to as an “ETP band”. In some embodiments, an ETP band may comprise one or more types of ions, analytes, and/or samples. In some instances, an ETP band may comprise a single type of analyte whose separation from other materials present in a sample is desired, e.g., separation of target nucleic acid from cellular debris. In some instances, an ETP band may contain more than one target analyte, e.g., polypeptides or nucleic acids sequences highly similar in sequence, e.g., allelic variants. In some instances, the ETP band may comprise different analytes of similar size or electrophoretic mobility. In such instances, the more than one target analyte may be separated by further ETP runs, e.g., under different conditions that promote separation of said more than one analyte, and/or said more than one analyte may be separated by other techniques known in the art for separation of analytes, such as those described herein. In some embodiments, an ETP band may be collected and optionally subject to further analysis after one or more ETP-based isolations/purifications and collections. In some embodiments, an ETP band may comprise one or more target analytes undergoing or that have undergone ETP-based isolation/purification and optionally collection, e.g., as a part of an ETP-run.

The term “target nucleic acid” as used herein is intended to mean any nucleic acid to be detected, measured, amplified, isolated, purified, and/or subject to further assays and analyses. A target nucleic acid may comprise any single and/or double-stranded nucleic acid. Target nucleic acids can exist as isolated nucleic acid fragments or be a part of a larger nucleic acid fragment. Target nucleic acids can be derived or isolated from essentially any source, such as cultured microorganisms, uncultured microorganisms, complex biological mixtures, samples including biological samples, urine samples, tissues, sera, ancient or preserved tissues or samples, environmental isolates or the like. Further, target nucleic acids include or are derived from cDNA, RNA, genomic DNA, cloned genomic DNA, genomic DNA libraries, enzymatically fragmented DNA or RNA, chemically fragmented DNA or RNA, physically fragmented DNA or RNA, or the like. In some embodiments, a target nucleic acid may comprise a whole genome. In some embodiments, a target nucleic acid may comprise the entire nucleic acid content of a sample and/or biological sample, e.g., a urine sample. Target nucleic acids can come in a variety of different forms including, for example, simple or complex mixtures, or in substantially purified forms. For example, a target nucleic acid can be part of a sample that contains other components or can be the sole or major component of the sample. Also, a target nucleic acid can have either a known or unknown sequence.

The term “target microbe” as used herein is intended to mean any unicellular or multicellular microbe, found in blood, plasma, other body fluids, samples such as biological samples, and/or tissues, e.g., one associated with an infectious condition or disease. Examples thereof include bacteria, archaea, eukaryotes, viruses, yeasts, fungi, protozoan, amoeba, and/or parasites. Furthermore, the term “microbe” generally refers to the microbe that may cause a disease, whether the disease is referred to or the disease-causing microbe is referred to.

As used herein, the term “biomarker” or “biomarker of interest” refers to a biological molecule found in tissues, blood, plasma, urine, and/or other body fluids that is a sign of a normal or abnormal process, or of a condition or disease (such as cancer). A biomarker may be used to see how well the body responds to a treatment for a disease or condition. In the context of cancer, a biomarker refers to a biological substance that is indicative of the presence of cancer in the body. A biomarker may be a molecule secreted by a tumor or a specific response of the body to the presence of cancer. Genetic, epigenetic, proteomic, glycomic, and imaging biomarkers can be used for cancer diagnosis, prognosis, and epidemiology. Such biomarkers can be assayed in non-invasively collected biofluids like blood, serum, and/or urine. Biomarkers may be useful as diagnostics (e.g., to identify early stage cancers) and/or prognostics (e.g., to forecast how aggressive a cancer is and/or predict how a subject will respond to a particular treatment and/or how likely a cancer is to recur).

The term “sample” as used herein includes a specimen or culture (e.g., microbiological cultures) that includes or is presumed to include one or more target analytes. The term “sample” is also meant to include biological, environmental, and chemical samples, as well as any sample whose analysis is desired. A sample may include a specimen of synthetic origin. A sample may include one or more microbes from any source from which one or more microbes may be derived. A sample may include, but is not limited, to whole blood, skin, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), tissue samples, biopsy samples, urine, feces, sputum, saliva, nasal mucous, prostate fluid, semen, lymphatic fluid, bile, organs, bone marrow, tears, sweat, breast milk, breast fluid, embryonic cells and fetal cells. In some instances, the sample may be a urine sample.

The term “target analyte” as used herein is intended to mean any analyte to be detected, measured, separated, concentrated, isolated, purified, and/or subject to further assays and analyses. In some embodiments, said analyte may be, but is not limited to, any ion, molecular, nucleic acid, biomarker, protein, cell, or population of cells, e.g., desired cells, and the like, whose detection, measurement, separation, concentration, and/or use in further assays is desired. In some embodiments, a target analyte may be derived from any of the samples described herein, e.g., a urine sample.

For purposes of the present disclosure, it will be understood that when a given component such as a layer, region, liquid or substrate is referred to herein as being disposed or formed “on”, “in” or “at” another component, that given component can be directly on the other component or, alternatively, intervening components (e.g., one or more buffer layers, interlayers, electrodes or contacts) can also be present. It will be further understood that the terms “disposed on” and “formed on” are used interchangeably to describe how a given component is positioned or situated in relation to another component. Hence, the terms “disposed on” and “formed on” are not intended to introduce any limitations relating to particular methods of material transport, deposition, or fabrication.

The term “communicate” is used herein to indicate a structural, functional, mechanical, electrical, optical, thermal, or fluidic relation, or any combination thereof, between two or more components or elements. As such, the fact that one component is said to communicate with a second component is not intended to exclude the possibility that additional components may be present between, and/or operatively associated or engaged with, the first and the second component.

As used herein, a “subject” refers to a mammalian subject (such as a human, rodent, non-human primate, canine, bovine, ovine, equine, feline, etc.) to be treated and/or one from whom a sample is obtained.

“Detecting” a sample within the context of an epitachophoresis device, system or machine may comprise detecting its position at one, several, or many points throughout the device. Detection may generally occur by any one or more means that do not interfere with desired device, system, or machine function and with methods performed using said device, system, or machine. In some embodiments, detection encompasses any means of electrical detection, e.g., through the detection of conductivity, resistivity, voltage, current, and the like. Furthermore, in some embodiments, detection may comprise any one or more of the following: electrical detection, thermal detection, optical detection, spectroscopic detection, photochemical detection, biochemical detection, immunochemical detection, and/or chemical detection. In some embodiments, one or more target analytes may be detected during ETP-based isolation/purification and optionally collection of said one or more target analytes. Moreover, sample detection within the context of ETP devices and methods of ETP are further described in U.S. Ser. Nos. 62/585,219 and 62/744,984; and PCT nos. PCT/EP2018/081049 and PCT/EP2019/077714, which disclosures are hereby incorporated by reference in their entirety herein.

In a sample analysis device or system, the term “sample collection volume” refers to a volume of sample intended for collection, e.g., by a robotic liquid handler, during or following analysis. In a device for effecting epitachophoresis, or a system comprising such a device, the sample collection volume is the volume intended for collection that comprises sample during or following epitachophoresis. In some embodiments, the sample collection volume may be located in the central well of a device or system described herein. In some embodiments, the sample collection volume may be located anywhere that permits collection of the desired sample. In some embodiments, the sample collection volume may be anywhere between the sample loading area and the leading electrolyte electrode/collection reservoir. The sample collection volume may be comprised by any suitable area, container, well, or space of the device or system. In some embodiments, the sample collection volume is comprised by a well, membrane, compartment, vial, pipette, or the like. In some embodiments, the sample collection volume may be formed by the space within or between components of the device or system, e.g. the space between two gels or a hole in a gel.

As used herein, the terms “ETP device”, “device for effecting ETP”, “device for ETP”, and the like, are used interchangeably to refer to devices which can perform, or on which can be performed, ETP and/or methods comprising ETP.

As used herein, the term “ETP-based isolation/purification” generally refers to devices and methods comprising ETP, e.g., devices on which ETP may be effected, e.g., methods comprising effecting ETP, wherein ETP focuses one or more target analytes into one or more focused zones (e.g., one or more ETP bands), thereby isolating/purifying the one or more target analytes from other materials comprised by an initial sample. It is noted the terms “isolate” and “purify” are used interchangeably. Furthermore, ETP based isolation/purification generally allows for subsequent collection of the one or more focused zones (one or more ETP bands) comprising said one or more target analytes. The degree of isolation/purification of one or more target analytes effected by one or more ETP-based isolations/purifications may be any degree or amount of isolation/purification of one or more target analytes from other materials. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of said target analyte, e.g., as measured by an analytical technique to determine the composition of an ETP isolated/purified sample comprising one or more target analytes. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of a target analyte being recovered from the original sample. In some embodiments, one or more ETP-based isolations/purifications may be effected to isolate/purify one or more target analytes, e.g., one or more nucleic acids. For example, in some instances, ETP-based isolation/purification may be effected on a sample comprising one or more target analytes to focus the one or more target analytes into one focused zone (ETP band), which substantially separates the one or more target analytes from other materials comprised in the original sample. The sample may be collected following ETP isolation/purification, and the isolated/collected sample may be further subject to another ETP-based isolation/purification. Optionally, the second ETP-based isolation-purification may be of such conditions so as to, in instances of more than one target analyte, isolate each of one or more target analytes into separate focused zones, each of which could optionally collected individually, thereby separating target analytes from one another, if desired.

As used herein, the term “mixed sample” generally refers to a sample comprising material from more than one source.

As used herein, the terms “sample pre-treatment”, “pre-treated sample”, and the like, generally refers to any procedures performed on said samples prior to loading the sample onto an ETP device. For example, in some instances a urine sample or other high salt comprising biological sample may be pre-treated by concentrating, buffering exchanging, and/or desalting said sample, such as by using centrifugation-based methods known in the art. For instance, a urine sample of any volume, e.g., from 1-50 mL, may be concentrated, desalted, and buffer exchanged using AMICON® Ultra Centrifugal filters. In some instances, desalting and/or buffer exchanging may be accomplished by use of dialysis-based methods. As a result of such procedures, an initial enrichment of the one or more target analytes, such as cfNAs, proteins, and/or extracellular vesicles, e.g., urinary exosomes, is accomplished, optionally in a desired buffer and/or in a lower salt solution as a result of the procedure. Moreover in some instances, a sample pre-treatment may include a filtration step, such as a vacuum filtration step, can be performed to remove unwanted materials, such as, for example, unwanted cells and/or unwanted cellular debris. Such a filtration step can be accomplished by using centrifugation-based filtration and/or vacuum-based filtration using a filter size selected to remove the unwanted materials, such as a 0.22 um filter membrane to remove said unwanted materials. Furthermore, in some instances, following sample concentration, desalting, and/or buffer exchange, the sample is subject to an extracellular vesicle enrichment step, such as an exosome enrichment step. Such enrichment steps can free circulating nucleic acids, e.g., DNAs and/or RNAs, from vesicles prior to introduction of the sample into the ETP device. Following any desired degree of sample pre-treatment, including any, all, or none of the above described procedures as well as other sample pre-treatment procedures well-known in the art, the sample may be loaded into an ETP device for one or more ETP runs to effect sample analysis. In some instances, following sample concentration and buffer exchange, a lysis and/or protein digestion step may be performed prior to introduction of the sample into the ETP device. Such steps may be effected at least in part based on the origin of target nucleic acids and/or the desired target nucleic acids to isolate/purify. Origins of nucleic acids found in urine include, for example, epithelial cells shed from the urinary tract, freely circulating cfDNA, and exosomes. In some instances, if total nucleic acid recovery is desired, a lysis step and/or protein digestion step may be performed using appropriate reagents. For example, proteinase K may be used to effect protein digestion. Moreover, such lysis and protein digestion steps may allow for all nucleic acids to be released from, for example, cells, exosomes, and histones (in case of cfDNA) prior to ETP-based isolation/purification. In some instances, nucleic acid recovery which does not include nucleic acids that originated from epithelial cells shed from the urinary tract is desired, a centrifugation step may be performed to effect removal of these epithelial cells prior to lysis and/or protein digestion steps. Following the centrifugation, the supernatant may be collected, while a pellet, which is presumed to contain the epithelial cells, may be discarded. The target nucleic acids that may be subsequently isolated/purified by ETP include cfDNA as well as nucleic acids encapsulated in exosomes. In some instances when nucleic acid recovery that targets cfDNA is desired, a protein digestion step, such as with proteinase K, may be performed prior to introducing the sample into an ETP device. No lysis step is performed, and furthermore any sources of unintentional lysis are avoided to prevent the release of nucleic acids from cells that may be present in the urine sample.

As used herein, the term “high salt comprising biological sample” generally refers to a biological sample recognized as comprising a salt concentration greater than that of other biological samples. For instance, a high salt comprising biological sample includes urine samples. In some instances, a human urine sample may comprise a urine sodium of about 10 mEq/liter. In some instances, a high salt comprising biological sample comprises sodium and/or potassium and/or calcium salts.

As used herein, the term “urine sample solution” generally refers to a urine sample that has underwent sample pre-treatment prior to being loaded into a device for effecting ETP to isolate/purify one or more nucleic acids comprised in the urine sample.

As used herein, the term “cell-free nucleic acid” (“cfNA”) generally refers to non-encapsulated nucleic acids that may be found in the urine and/or bloodstream of an organism. In some instances, cfNA may be isolated from a urine, blood, plasma, and/or serum sample, and the like. In some instances, the cell-free nucleic acid may be cell-free DNA (cfDNA). In some instances, the cell-free nucleic acid may be cell-free RNA (cfRNA). In some instances, the cell-free nucleic acids may be a mixture of cell-free DNA (cfDNA) and cell-free RNA (cfRNA). In some instances, cfNA may comprise fetal DNA and/or maternal DNA. In some instances, a urine sample from a pregnant woman may comprise cfNA. In some instances, cfNA may comprise circulating tumor nucleic acids (ctNA). In some instances, cfNA may comprise DNA and/or RNA fragments of about 1000 bp or more, 1000 bp or less, 900 bp or less, 800 bp or less, 700 bp or less, 600 bp or less, 500 bp or less, 400 bp or less, 300 bp or less, 250 bp or less, 200 bp or less, 150 bp or less in length, e.g., about 180 bp or less. In some instances, cfNA may be isolated/purified and optionally collected using the ETP-based devices and methods described herein. In some embodiments, following ETP-based isolation/purification, the isolated/purified cfNA may be collected and may be subjected to any one or more of the further analytical techniques described herein, e.g., sequencing e.g., one or more IVD assays.

As used herein, the term “circulating tumor nucleic acid NA” (ctNA) refers to cfNA that originates from a cancerous cell, e.g., a tumor cell. In some instances, ctDNA may enter the bloodstream during apoptosis or necrosis of a cancerous cell. In some instances, this ctDNA may then enter the urine by passing through the kidneys. In some instances, the tumor nucleic acid may be a circulating tumor RNA (ctRNA). In some instances, the circulating tumor nucleic acid may be a circulating tumor DNA (ctDNA). The ctNA may be a mixture of ctDNA and ctRNA. In some embodiments, ctNA may be isolated/purified and optionally collected using the ETP-based devices and methods described herein. In some embodiments, following ETP-based isolation/purification, isolated/purified ctNA may be collected and may be subjected to any one or more of the further analytical techniques described herein, e.g., sequencing, e.g., one or more IVD assays. In some instances, ctNA may comprise DNA fragments and/or RNA fragments of about 1000 bp or more, 1000 bp or less, 900 bp or less, 800 bp or less, 700 bp or less, 600 bp or less, 500 bp or less, 400 bp or less, 300 bp or less, 250 bp or less, 200 bp or less, 150 bp or less in length, e.g., about 150 bp in length.

As used herein, the term “ETP upper marker” generally refers to a compound or molecule that is larger in size and/or longer in length as compared to a target nucleic acid, such that, during ETP-based isolation/purification and subsequent collection of a target analyte, the ETP upper marker indicates a cutoff point at which collection of the target analyte can be stopped. For example, fluorescently labeled, or otherwise detectably labeled, ETP upper maker can be generated of such a size that it is larger than a target DNA to be isolated/purified and collected during ETP-based isolation/purification. By monitoring the marker throughout the ETP run, the user or automated machine is able to stop the run before the marker falls into a collection tube, thereby allowing DNA smaller than the marker to be captured while larger contaminating DNA are left out as they are positioned behind the upper marker. Moreover, the ETP upper marker itself is not collected, and as such can be used at high quantity and with various detectable labels since it will not interfere with downstream assays, e.g., one or more IVD assays. In some instances, an ETP upper marker may be used in ETP-based isolation/purification methods as it aids in the exclusion of genomic DNA from an isolated/purified and collected sample of one or more target analytes.

As used herein, the term “extracellular vesicles” generally refers to cell-derived vesicles (membrane enclosed bodies) present in biological fluids, e.g., urine. Examples of extracellular vesicles include exosomes, microvesicles, and apoptotic bodies. Extracellular vesicles can be released from cells, e.g., directly from the plasma membrane, or formed when multivesicular bodies fuse with the plasma membrane. Extracellular vesicles typically include components such as nucleic acids and/or proteins from their cell of origin. Exosomes are typically 40-120 nm in diameter, microvesicles are typically 50-1000 nm in diameter, and apoptotic bodies are typically 500-2000 nm in diameter.

### DETAILED DESCRIPTION

As noted above, current approaches to extracting, isolating, and/or purifying nucleic acids from urine samples, have many drawbacks. For example, one such drawback is that the concentration of biomarker, e.g., nucleic acid, can be relatively low, so that a large volume is needed to obtain sufficient material for downstream analysis. In the case of nucleic acids, conventional techniques using nucleic acid capture kits and devices are generally designed for small sample volumes, which are typically in the range of 0.2-1 mL. As such, in spite of being a biomarker-rich resource, efficient isolation and purification of biomarkers from urine has thus far proven challenging. Moreover, other disadvantages associated with conventional techniques for the extraction, isolation and/or purification of biomarkers from urine include often time-consuming procedures that yield low quality product, and the lack of suitability for automation. Conventional techniques may include a series of affinity columns to extract certain fractions from the urine sample. The fractions may then be combined before being sent for further analysis. Such column-based techniques are not high throughput. As an example, 10 mL of urine may need to go through multiple (e.g., 10) column cleanups.

To solve such problems, the present disclosure generally describes devices and methods for sample analysis, e.g., analysis of urine samples, comprising one or more target analytes, wherein said devices and methods comprise effecting epitachophoresis to isolate and/or purify said target analytes from said sample, wherein the isolated and/or purified nucleic acids optionally may be subjected to further downstream assays, such as in vitro diagnostic (“IVD”) assays. Moreover, it is noted that the highly efficient extraction of target nucleic acids obtained through the use of the devices and methods described herein is helpful for downstream in vitro diagnostic (IVD) methods, in which the amount of target nucleic acid, e.g., DNA and/or RNA, directly correlates with the sensitivity that may be achieved in said down-stream IVD assay, a significant advantage over current methodologies. For example, spin columns or magnetic glass particles that bind nucleic acids on their surface conventionally are used in order to effect extraction of nucleic acids. As compared to such conventional approaches, the devices and methods described herein may confer any one or more of the following advantages: higher extraction yields (potentially loss-less) compared to column- or bead-based extraction methods; a simpler device setup compared to the larger footprint for benchtop instruments; potentially faster sample turn-around and high parallelizability as compared to other devices applied to similar uses; easy integration with other microfluidics-based systems for down-stream processing of isolate/purified target analytes, e.g., biomarkers, e.g., nucleic acids. In some embodiments, the target analytes obtained by ETP-based isolation/purification of a sample, e.g., a urine sample, may comprise the total nucleic acid content of said sample, e.g., both DNA and RNA from said sample. In some instances, methods comprising ETP-based isolation/purification may comprise simultaneous collection of nucleic acids comprising DNA and RNA, and the collected nucleic acids may be subjected to methods for separating the DNA and RNA for further downstream assays, e.g., separation of DNA and RNA by any means known in the art.

Moreover, the present disclosure generally relates to a method of isolating and/or purifying one or more target analytes from a sample, such as a urine or other high salt comprising biological sample, e.g., sodium and/or potassium salts, wherein said method comprises: a. providing a device for effecting epitachophoresis (ETP); b. providing a sample potentially comprising said one or more target analytes, optionally one or more target nucleic acids; c. performing one or more epitachophoresis runs by effecting ETP using said device to focus said one or more target analytes into one or more focused zones, e.g., as one or more ETP bands; and d. collecting said one or more target analytes by collecting said one or more focused zones comprising said one or more target analytes; thereby obtaining one or more isolated and/or purified target analytes, optionally wherein said sample comprises nucleic acids, further optionally wherein the sample comprises a urine sample and prior to step c. the urine sample is subject to one or more pre-treatments. For example, in some instances a urine sample may be pre-treated by concentrating, buffering exchanging, and/or desalting said urine sample, such as by using centrifugation-based methods known in the art.

As ETP is used for concentrating a sample, one of skill in the art would not expect to perform a further concentration step on a urine sample or other sample before ETP. Running a non-concentrated urine sample through ETP would not result in concentrated analytes. Certain components (e.g., salts) in the urine sample are incompatible with ETP. Additionally, one of skill in the art may consider concentrating urine samples with affinity columns. If such columns were used for some amount of concentration, one of skill in the art would not be motivated to use ETP in combination with affinity columns as the affinity columns would limit throughput and may limit advantages of ETP. One would therefore not be motivated to concentrate a sample prior to ETP. Furthermore, concentrating urine sample may not be expected to use the techniques (e.g., centrifugation) and systems described herein.

For instance, a urine sample of any volume, e.g., from 1-50 mL, may be concentrated, desalted, and buffer exchanged using AMICON® Ultra Centrifugal filters (e.g., Example 9 below). In some instances, desalting and/or buffer exchanging may be accomplished by use of dialysis-based methods. As a result of such procedures, an initial enrichment of the one or more target analytes, such as cfNAs, proteins, and/or extracellular vesicles, e.g., urinary exosomes, is accomplished, optionally in a desired buffer and/or in a lower salt solution as a result of the procedure. Moreover in some instances, a sample pre-treatment may include a filtration step, such as a vacuum filtration step, can be performed to remove unwanted materials, such as, for example, unwanted cells and/or unwanted cellular debris. Such a filtration step can be accomplished by using centrifugation-based filtration and/or vacuum-based filtration using a filter size selected to remove the unwanted materials, such as a 0.22 um filter membrane to remove said unwanted materials. Furthermore, in some instances, following sample concentration, desalting, and/or buffer exchange, the sample is subject to an extracellular vesicle enrichment step, such as an exosome enrichment step. Such enrichment steps can free circulating nucleic acids, e.g., DNAs and/or RNAs, from vesicles prior to introduction of the sample into the ETP device. In some instances, following sample concentration and buffer exchange, a lysis and/or protein digestion step may be performed prior to introduction of the sample into the ETP device. Such steps may be effected at least in part based on the origin of target nucleic acids and/or the desired target nucleic acids to isolate/purify. Origins of nucleic acids found in urine include, for example, epithelial cells shed from the urinary tract, freely circulating cfDNA, and exosomes. In some instances, if total nucleic acid recovery is desired, a lysis step and/or protein digestion step may be performed using appropriate reagents. For example, proteinase K may be used to effect protein digestion. Moreover, such lysis and protein digestion steps may allow for all nucleic acids to be released from, for example, cells, exosomes, and histones (in case of cfDNA) prior to ETP-based isolation/purification. In some instances, nucleic acid recovery which does not include nucleic acids that originated from epithelial cells shed from the urinary tract is desired, a centrifugation step may be performed to effect removal of these epithelial cells prior to lysis and/or protein digestion steps.

Following the centrifugation, the supernatant may be collected, while a pellet, which is presumed to contain the epithelial cells, may be discarded. The target nucleic acids that may be subsequently isolated/purified by ETP include cfDNA as well as nucleic acids encapsulated in exosomes. In some instances when nucleic acid recovery that targets cfDNA is desired, a protein digestion step, such as with proteinase K, may be performed prior to introducing the sample into an ETP device. No lysis step is performed, and furthermore any sources of unintentional lysis are avoided to prevent the release of nucleic acids from cells that may be present in the urine sample. Following any desired degree of sample pre-treatment, including any, all, or none of the above described procedures as well as other sample pre-treatment procedures well-known in the art, the sample may be loaded into an ETP device for one or more ETP runs to effect sample analysis.

In some embodiments, the method of isolating and/or purifying one or more target analytes, e.g., nucleic acids, from a sample, such as a urine or other high salt comprising biological sample, e.g., sodium or potassium salts, comprises: a. providing a device for effecting epitachophoresis (ETP); b. providing a high salt comprising biological sample, optionally a urine sample, potentially comprising one or more target analytes; c. effecting one or more pre-treatment steps on said urine sample; d. performing one or more epitachophoresis runs by effecting ETP using said device to focus said one or more target analytes into one or more focused zones, e.g., as one or more ETP bands; and e. collecting said one or more target analytes by collecting said one or more focused zones comprising said one or more nucleic acids; thereby obtaining one or more isolated and/or purified target analytes. In some instances, the target analytes may comprise one or more nucleic acids, e.g., one or more cfNAs.

In some embodiments, said method for ETP-based isolation and/purification of one or more target analytes may be automated, e.g., by using an automated ETP system. See, for instance, U.S. Patent Publication No. US 2020/0282392, filed Nov. 13, 2018; U.S. Application Ser. No. 62/744,984, filed Oct. 12, 2018; U.S. Application Ser. No. 62/847,678, filed May 14, 2019; PCT Patent Publication No. WO 2019/092269, filed Nov. 13, 2018; PCT Patent Publication No. WO 2020/074742, filed Oct. 14, 2019, which disclosures are hereby incorporated by reference in their entirety herein.

In some embodiments, an ETP device for use with the methods described herein may comprise an ETP device as described in U.S. Patent Publication No. US 2020/0282392, filed Nov. 13, 2018; U.S. Application Ser. No. 62/744,984, filed Oct. 12, 2018; U.S. Application Ser. No. 62/847,678, filed May 14, 2019; PCT Patent Publication No. WO 2019/092269, filed Nov. 13, 2018; PCT Patent Publication No. WO 2020/074742, filed Oct. 14, 2019, which disclosures are hereby incorporated by reference in their entirety herein.

In some embodiments, said method may comprise ETP-based isolation and/or purification of one or more nucleic acids from one or more urine samples, wherein said nucleic acids comprise cfNAs, e.g., cfDNA and/or cfRNA. In some instances, said method may result in isolating and/or purifying both DNA and RNA in one or more ETP bands and/or focused zones during a single ETP run. In some instances, following ETP-based isolation and/or purification, RNA and DNA may be separated from one another and the RNA and/or DNA may be subjected to further downstream assays, such as one or more IVD assays, sequencing, and/or gene expression profiling. In some embodiments, said urine samples may comprise a urine sample solution resulting from one or more pre-treatments of the urine samples.

In some embodiments, said method may comprise ETP-based isolation and/or purification of one or more nucleic acids from one or more urine samples, wherein said nucleic acids originate from one or more cells, such as, for example, epithelial cells, leukocytes, malignant cells, and/or any other cells that may be present in urine, such as those spontaneously liberated into urine.

In some embodiments, said method may comprise ETP-based isolation and/or purification of one or more biomarkers from one or more urine samples, wherein said biomarkers may include any one or more of nucleic acids, cfNAs, proteins, and/or extracellular vesicles. In some instances, said method may result in isolating and/or purifying any of the one or more biomarkers in one or more ETP bands and/or focused zones during a single ETP run. In some instances, following ETP-based isolation and/or purification, the one or more biomarkers may be separated from one another prior to further downstream assays, such as one or more IVD assays, sequencing, and/or gene expression profiling. In some embodiments, said urine samples may comprise a urine sample solution resulting from one or more pre-treatments of the urine samples.

In some embodiments, a device and/or method for epitachophoresis may focus and allow for collection of a target analyte in any desired amount of time that allows for a desired focusing and collection to occur. In some embodiments, said method may comprise effecting ETP for 120 minutes or more, 120 minutes or less, 100 minutes or less, 80 minutes or less, 60 minutes or less, 50 minutes or less, or 40 minutes or less.

In some embodiments, the target analytes, e.g., nucleic acids, obtained by ETP-based isolation/purification of nucleic acids from urine samples may be of a higher yield and/or higher quality as compared to target analytes obtained from urine samples using conventional techniques, such as those described supra, e.g., bead-based and/or column based methods. In some embodiments, target analytes obtained by ETP-based isolation/purification of nucleic acids from one or more urine samples may be of an equal or higher quality as measured an applicable technique, such as by qPCR-based analysis, e.g., a Q score obtained from said qPCR-based analysis such as quality control (qc) qPCR. It is noted that Q scores range from 0 (low quality) to 1 (high quality), and higher quality samples are generally preferred for downstream IVD applications, such as sequencing-based applications. In some embodiments, 1.25 times or more, 1.5 times or more, 1.75 times or more, 2.0 times or more, 2.25 times or more, 2.5 times or more, 2.75 times or more, 3 time or more, 4 times or more, 5 times or more, 10 times or more, 100 times or more, or 1000 times or more target analytes may be obtained using said a method comprising ETP-based isolation/purification of nucleic acids from one or more urine samples as compared to conventional methods such as those comprising a bead-based and/or column-based method. In some embodiments, the amount of isolated and/or purified nucleic acids obtained from the methods described herein may be any amount and may at least in part on the sample used. In some instances, the amount of isolated and/or purified nucleic acids may range anywhere from a nanogram or less to micrograms or more and/or macrograms or more.

In some embodiments, a device for sample analysis may comprise a gel, or other material which may be used to stabilize a leading electrolyte. In further embodiments, a device for sample analysis may comprise a gel, and said gel may help to avoid unwanted sample contamination. For example, a device for sample analysis may be used to extract ctDNA, and said gel may be used to help avoid contamination of ctDNA with genomic DNA and/or cellular debris. To avoid said unwanted contamination, the gel may be of such a composition so as to allow ctDNA, but not genomic DNA or cellular debris, to migrate through said gel. Such a principle may be applied to other sample analyses where it may be beneficial to avoid contamination of a sample of interest/target analyte. In some embodiments, mesh polymers and/or porous materials may be used in a similar manner as to a gel in devices for sample analysis, such as, for example, filter paper or hydrogels. The selection of said mesh polymer and/or porous material may be that which helps to effect a desired separation/concentration and/or to prevent undesired sample contamination. For example a material may be selected that does not permit passage/migration of proteins but can allow passage/migration of target nucleic acids.

In some embodiments, a device for sample analysis may be used to focus and collect tumor DNA and/or circulating tumor DNA (ctDNA), and/or circulating cfDNA, e.g., those present in urine from pregnant women, and/or circulating DNAs expressing proteins over or under expressed in specific conditions which may then optionally be subjected to further downstream analyses, such as nucleic acid sequencing and/or other in vitro diagnostic applications. Such downstream in vitro applications include by way of example disease detection such as cancer diagnosis and/or cancer prognosis and/or cancer staging, detection of infectious conditions, paternity analysis, detection of fetal chromosomal abnormalities such as aneuploidy, detection of fetal genetic traits, detection of pregnancy-related conditions, detection of autoimmune or inflammatory conditions, among a myriad of other potential uses.

In some embodiments, a method for sample analysis may comprise focusing and collecting a target nucleic acid, and said target nucleic acid may be of any desired size. For example, said target nucleic acid may be 5 nt or less, 10 nt or less, 20 nt or less, 30 nt or less, 50 nt or less, 100 nt or less, 1000 nt or less, 10,000 nt or less, 100,000 nt or less, 1,000,000 nt or less, or 1,000,000 nt or more.

Furthermore, the present disclosure generally relates to devices and methods comprising ETP-based isolation/purification of one or more target analytes, e.g., one or more target nucleic acids, which may comprise cell-free nucleic acids (cfNA), e.g., cfDNA, by providing a device for effecting ETP; providing a sample, e.g., a urine sample, e.g., a urine sample solution, comprising said one or more cell-free nucleic acids; performing one or more ETP runs by effecting ETP using said device, wherein said ETP run focuses said one or more cfNAs into one or more focused zones, e.g., as one or more ETP bands; and collected said one or more cfNAs, thereby obtaining one or more isolate/purified cfNAs.

In some embodiments, cfNA, e.g., cfDNA, isolated/purified by ETP-based methods and devices, may be further subject to an assay system that utilizes both non-polymorphic and polymorphic detection to determine source contribution and copy number variations (CNVs) from a single source within a mixed sample, e.g., urine sample, such as that described in U.S Patent Application Publication No. 2012/0034685, which is hereby incorporated by reference in its entirety.

Moreover, the present disclosure generally relates to the isolation/purification of one or more cfNAs, e.g., one or more cfDNAs and/or one or more cfRNAs, by ETP-based devices and methods from one or more samples, e.g., a urine sample, wherein said isolated/purified one or more cfNAs are further analyzed to detect fetal aneuploidy. For example, such an assay to detect fetal aneuploidy is generally described in U.S Patent Application Publication No. 2012/0034685, which is hereby incorporated by reference in its entirety, and in particular as described in Example 7 of the cited reference.

In addition to the detection of aneuploidy, specific polymorphisms may also be used to determine percent fetal contribution to the maternal samples, e.g., urine sample, wherein cfNAs, e.g., cfDNAs, isolated/purified by ETP-based devices and methods described herein is used in such determinations. The general methodology used for determination of these fetal contribution percentages is described in U.S. Patent Publication No. 2013/0024127 A1, filed Jul. 19, 2012, which is incorporated by reference in its entirety.

Furthermore, the use of cfNA, e.g., cfDNA, isolated/purified from a sample, such as a urine sample, by ETP-based methods and devices as discussed herein may further be subject to CNV analysis, such as that described above, which may allow the identification of both CNVs and infectious agents in a mixed sample.

Furthermore, cfNA, e.g., cfDNA, isolated/purified from samples such as urine samples by ETP-based devices and methods such as those described herein, may further be subject to the detection of quantitative and qualitative tumor-specific alterations of the cfNA, such as cfDNA strand integrity, frequency of mutations, abnormalities of microsatellites, and methylation of genes, as diagnostic, prognostic, and monitoring markers in cancer patients, and may optionally be combined with CNV detection to provide a method for assisting with clinical diagnosis, treatments, outcome prediction and progression monitoring in patients with or suspected of having a malignancy. For further discussion, see U.S Patent Application Publication No. 2012/0034685, which is hereby incorporated by reference in its entirety herein.

Furthermore, cfNA, e.g., cfDNA, isolated/purified from a sample, e.g., a urine sample, by ETP-based devices and methods such as those described herein may be further subject to assay systems of that can be used to monitor organ health in a transplant patient using a combination of detection of cfDNA and detection of SNPs or mutations in one or more single genes (see U.S Patent Application Publication No. 2012/0034685 for further discussion, incorporated herein in its entirety). Transplanted organs have genomes that are distinct from the genome of a recipient patient, and organ health can be detected using such an assay system. For example, acute cellular rejection has been shown to be associated with significantly increased levels of cell-free DNA from the donor genome in heart transplant recipients.

In some instances, target analyte, e.g., cfNA, isolated and collected from a sample, such as a urine sample, by ETP-based devices and methods may be subject to any one or more of the methods and/or assays of U.S Patent Application Publication No. 2012/0034685 (incorporated by reference in its entirety herein), such as, for example, in addition to the above methods and assays, those methods and assays described in the sections entitled “Assay Methods”; “Detecting Copy Number Variations”; “Polymorphisms Associated with Diseases or Predispositions”; “Selected Amplification”; “Universal Amplification”; “Variation Minimization within and Between Samples”; “Use of Assay Systems for Detection in Mixed Samples from Cancer Patients”; “Use of Assay Systems for Detection of Mixed Samples from Transplant Patients”; “Use of Assay Systems for Detection in Maternal Samples”; and “Determination of Minor Source DNA Content in a Mixed Sample”.

Moreover, the present disclosure further generally encompasses a method of isolating/purifying one or more target analytes, e.g., one or more target nucleic acids, which method comprises ETP-based isolation/purification of said one or more target analytes which further comprises the use of an ETP upper marker. In some embodiments, said ETP upper marker may comprise a compound or molecule is larger in size and/or longer in length as compared to a target analyte, such that, during ETP-based isolation/purification of a target analyte, the ETP upper marker indicates a cutoff point at which collection of the target analyte can be stopped. For example, fluorescently labeled, or otherwise detectably labeled, ETP upper maker can be generated of such a size that it is larger than a target DNA to be collected during ETP-based isolation/purification. By monitoring the marker throughout the ETP run, the user or automated machine is able to stop the run before the marker falls into a collection tube, thereby allowing DNA smaller than the marker to be captured while larger contaminating DNA are left out as they are positioned behind the ETP upper marker. Moreover, the marker itself is not collected, and as such can be used at high quantity and wither various detectable labels since it will not interfere with downstream assays, e.g., IVD assays. In some instances, an ETP upper marker may be used in ETP-based isolation/purification methods as it aids in the exclusion of unwanted materials, e.g., exclusion of genomic DNA from isolated/purified cfDNA. In some embodiments, an ETP upper marker may be about 1000 bp or more in length.

Moreover, the present disclosure generally encompasses ETP-based isolation/purification of ctNA, e.g., ctDNA, from a sample such as a urine sample, wherein said ctNA may be further subject to methods comprising CAncer Personalized Profiling by Deep Sequencing (CAPP-Seq), such as described by U.S. Patent Application Publication No. 20160032396, which is hereby incorporated by reference in its entirety.

In some embodiments, in order to cause movement of the charged particles in the present methods and devices, within a convenient time frame, the electric field strength may be about 10 V to about 10 kV with electric powers ranging from about 1 mW to about 100 W. In some embodiments, the maximum electric power applied for the fastest analysis may depend on the electric resistivity of the sample and electrolyte solutions and the cooling capabilities of the materials that may be used for construction of the devices described herein.

In some embodiments, said ETP-based isolation and/or purification of one or more nucleic acids from one or more urine samples may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of the one or more target analytes comprised in the original sample being isolated and collected. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of said one or more target analytes isolated/purified, e.g., as measured by an analytical technique to determine the composition of an ETP isolated/purified sample comprising one or more target analytes. In some embodiments, one or more of the buffer concentrations, e.g., LE and/or TE buffer concentrations; percentage of a gel comprised in said ETP device, and/or the stoppage time of the ETP-based isolation and collection run may be varied and/or optimized to enhance separation of said one or more nucleic acids from other materials comprised in the sample.

In some embodiments, the one or more nucleic acids to be isolated/purified by ETP-based isolation/purification may be any desired size. In some embodiments, the nucleic acids may be 5 nt or less, 10 nt or less, 20 nt or less, 30 nt or less, 50 nt or less, 100 nt or less, 1000 nt or less, 10,000 nt or less, 100,000 nt or less, 1,000,000 nt or less, or 1,000,000 nt or more in size. In some embodiments, said method further may comprise detection of said one or more nucleic acids during and/or after said ETP-based isolation and/or purification, e.g., said detection comprises optical detection, in some instances, wherein said optical detection comprises detection of an intercalating dye and/or an optical label which binds to and/or is associated with said one or more nucleic acids. In some embodiments, said detection may comprise electrical detection, e.g., voltage monitoring. In some embodiments, detection may comprise monitoring the movement of a dye, e.g., Brilliant Blue, and adjusting any one or more ETP parameters, e.g., starting or stopping sample collection, based on the movement of said dye.

In some embodiments, the method of ETP-based isolation/purification of one or more target analytes, optionally from one or more urine samples, may be an automated method wherein the sample is automatically loaded into said device, and/or said one or more target analytes are automatically collected from said device. In some embodiments, the one or more isolated and/or purified target analytes may be subject to one or more further ETP runs to further isolate and/or purify said one or more target analytes.

Furthermore, the present disclosure generally relates to a method of identifying tumor-derived SNVs comprising (a) obtaining a sample from a subject suffering from a cancer or suspected of suffering from a cancer, optionally wherein the sample is an urine sample, e.g., urine sample solution; (b) performing ETP-based isolation and/or purification to isolate and/purify target nucleic acids, to obtain an isolated and/or purified sample; (c) conducting a sequencing reaction on the isolated and/or purified sample to produce sequencing information; (d) applying an algorithm to the sequencing information to produce a list of candidate tumor alleles based on the sequencing information from step (c), wherein a candidate tumor allele comprises a non-dominant base that is not a germline SNP; and (e) identifying tumor-derived SNVs based on the list of candidate tumor alleles. In some embodiments, the candidate tumor allele may comprise a genomic region comprising a candidate SNV.

Furthermore, the present disclosure generally relates to a method of identifying viral-derived nucleic acids comprising (a) obtaining a sample, e.g., a urine sample, from a subject suspected to have a virus infection or suspected of having been exposed to a virus; (b) performing ETP-based isolation and/or purification to isolate and/or purify target nucleic acids to obtain an isolated and/or purified nucleic acids; (c) conducting a sequencing reaction on the isolated and/or purified nucleic acids to produce sequencing information; and (d) determining based on the sequencing information whether the subject has been infected with one or more viruses.

Moreover, in further exemplary embodiments, devices for sample analysis as described herein may comprise dimensions that accommodate 0.25 mL or less, 0.25 mL or more, 0.5 mL or more, 0.75 mL or more, 1.0 mL or more, 2.5 mL or more, 5.0 mL or more, 7.5 mL or more, 10.0 mL or more, 12.5 mL or more, or 15.0 mL or more, 20.0 mL or more, 25.0 mL or more, 30.0 mL or more, 40.0 mL or more, or 50 mL or more of sample volume. In some embodiments, the concentration of the one or more isolated and/or purified nucleic acids may be measured. In some embodiments, the sample volume of the sample to be loaded into an ETP device for ETP-based isolation/purification may be 0.25 mL or less, 0.25 mL or more, 0.5 mL or more, 0.75 mL or more, 1.0 mL or more, 2.5 mL or more, 5.0 mL or more, 7.5 mL or more, 10.0 mL or more, 12.5 mL or more, or 15.0 mL or more, 20.0 mL or more, 25.0 mL or more, 30.0 mL or more, 40.0 mL or more, or 50 mL or more. In some embodiments, the sample volume may be 0.25 mL or less, 0.25 mL or more, 0.5 mL or more, 0.75 mL or more, 1.0 mL or more, 2.5 mL or more, 5.0 mL or more, 7.5 mL or more, 10.0 mL or more, 12.5 mL or more, or 15.0 mL or more, 20.0 mL or more, 25.0 mL or more, 30.0 mL or more, 40.0 mL or more, or 50 mL or more.

In further exemplary embodiments, said device may be used to concentrate a target analyte, e.g., from about 2 fold or more to about 1000 fold or more. In some embodiments, said target analyte may comprise one or more nucleic acids. In further embodiments, said target analyte may comprise small inorganic and organic ions, peptides, proteins, polysaccharides, DNA, or microbes such as bacteria and/or viruses. In some embodiments, said target analytes may comprise extracellular vesicles, e.g., urinary exosomes.

In some embodiments, said nucleic acids collected by ETP-based isolation/purification may be used for one or more downstream in vitro diagnostic applications. Furthermore, in some embodiments the ETP device for sample analysis may be connected on-line to other devices, such as, for example, capillary analyzers, chromatography, PCR devices, enzymatic reactors, and the like, and/or any other device that may be used to effect further sample analysis, e.g., a device associated with IVD applications. In some embodiments, the ETP device may be used in a workflow with nucleic acid sequencing library preparation. Moreover, in some embodiments, the ETP device may be used with liquid handling robots that may optionally be used to effect downstream analysis of a sample that may have been focused and/or collected from said device.

In some embodiments, the sample may comprise a urine sample comprising one or more biomarkers, e.g., one or more ctNAs, e.g., one or more proteins, associated with one or more types of cancer. In some embodiments the cancer comprises a cancer selected from the group consisting of acute lymphoblastic leukemia (ALL), acute myeloid leukemia (AML), Adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, Burkitt lymphoma, carcinoid tumors (e.g., childhood, gastrointestinal), cardiac tumors, cervical cancer, chordoma, chronic lymphocytic leukemia (CLL), chronic myelogenous leukemia (CIVIL), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, Langerhans cell cancer, Hodgkin lymphoma, hypopharyngeal cancer, intraocular melanoma, islet cell tumors, pancreatic neuroendocrine tumors, Kaposi sarcoma, kidney cancer (e.g., renal cell, Wilm's tumor, and other kidney tumors), Langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sezary syndrome), Hodgkin, non-Hodgkin, primary central nervous system (CNS)), macroglobulinemia, Waldenstrom, male breast cancer, malignant fibrous histiocytoma of bone and osteosarcoma, melanoma (e.g., childhood, intraocular (eye)), Merkle cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhabdomyosarcoma, soft tissue, uterine), Sezary syndrome, skin cancer (e.g., melanoma, Merkle cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenstrom macroglobulinemia, Wilm's tumor, and the like.

The devices and methods illustratively disclosed herein suitably may be practiced in the absence of any element which is not specifically disclosed herein and/or any element specifically disclosed herein.

### Devices for Electrophoresis

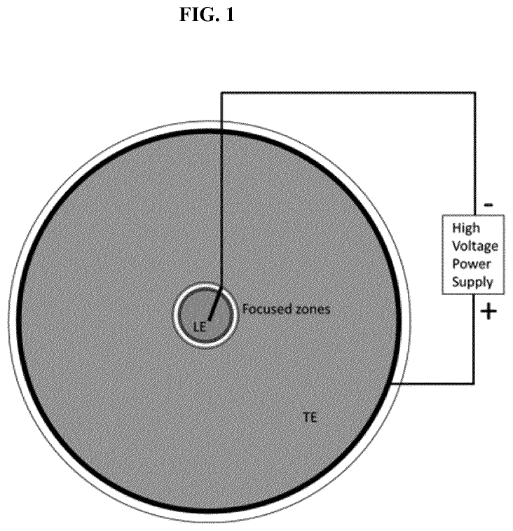

Devices for epitachophoresis generally use a concentric or polygonal disk architecture, for example, as depicted in FIG. 1-FIG. 4. Glass or ceramics are used for fabrication of the system (i.e. material for concentric or polygonal disks) as these materials result in improved heat transfer properties that are beneficial during device operation. For example, as the flat channel of a epitachophoresis device has a favorable heat transfer capability compared to a narrow channel, over-heating (or boiling) of the focused material is generally prevented. Current/voltage programming is also suitable for adjusting the Joule heating of the device. Plastic materials are also used for device fabrication. In general, devices are fabricated of such dimensions that accommodate a desired sample volume, such as milliliter-scale sample volumes, for example, up to 15 mL.

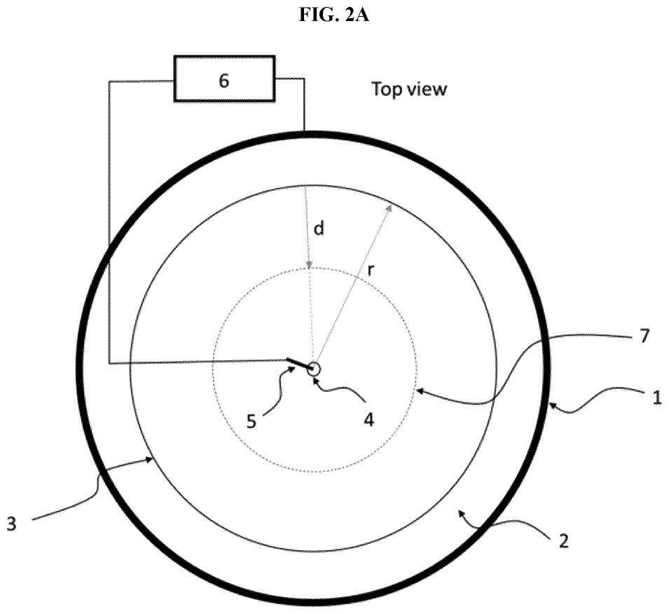

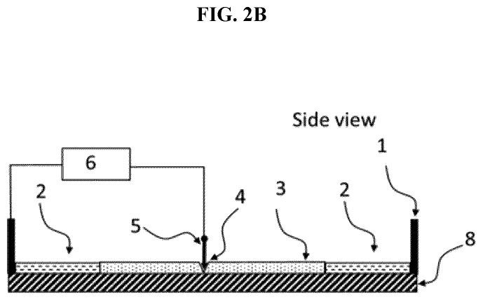

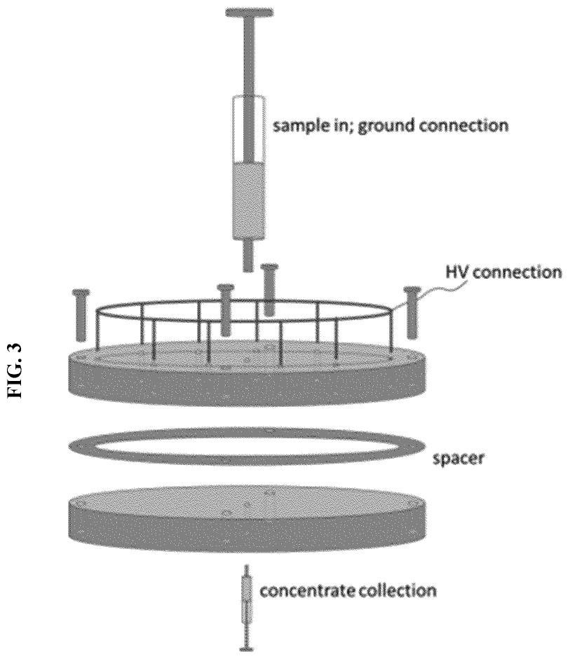

Referring to FIG. 1-FIG. 3, two concentric disks are separated by a spacer, thereby forming a flat channel for epitachophoresis sample processing. Electric current is applied through multiple high voltage connections (HV connection) and the ground connection in the center of the system (see FIG. 1 and FIG. 3, for example). In some instances, the sample is injected into the device through an opening in the device, e.g., in the top or the side (see, for example, FIG. 3). Application of electricity focuses the target analyte of a sample as a concentric ring that migrates to the center of the disk (discussed further below), and the target analyte is then collected through a syringe at the bottom of the device (see, for example, FIG. 3). As presented in FIG. 2A (top view) and FIG. 2B, an example of a device setup contains an outer circular electrode (1), terminating electrolyte (2), and leading electrolyte (3). In general, the diameter of the outer circular electrode (1) is about 10-200 mm and the diameter of the leading electrolyte ranges from a thickness (height) of about 10 μm to about 20 mm. The leading electrolyte is stabilized by a gel, viscous additive, or otherwise hydrodynamically separated from the terminating electrolyte, such as, for example, by a membrane. The gel or hydrodynamic separation prevents mixing of the leading and terminating electrolytes during device operation. Also, in some devices mixing is prevented by using very thin (<100 um) layers of electrolytes, as is discussed further below.

Referring to FIG. 2A-FIG. 2B, in the center of the leading electrolyte is an electrode reservoir (4) with electrode (5). The assembly of the electrodes (1, 5) and electrolytes (2, 3) is placed on a flat, electrically insulating support (8). The electrolyte reservoir (4) is used for removal of the concentrated sample solution following a separation process, such as by pipetting the sample out of the reservoir, for example. Electrode reservoir (4) is also a sample collection reservoir. Outer circular electrode (1) may be disposed at the end of a circular channel in which the leading electrolyte (3) and terminating electrolyte (2) are disposed.

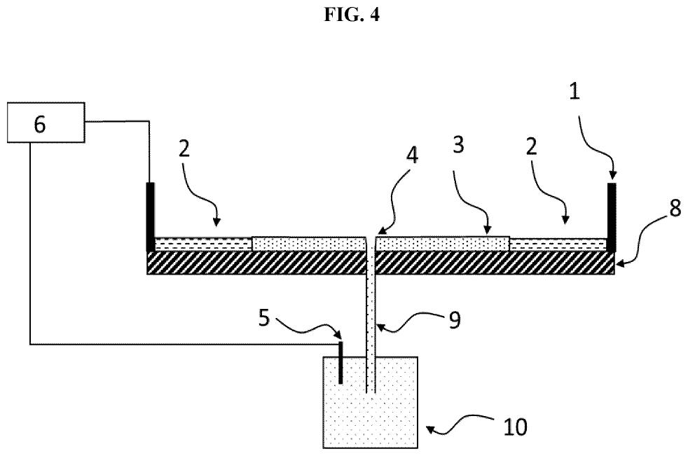

In an alternative arrangement (see FIG. 4) the center electrode (5) is moved to a leading electrolyte reservoir (10) connected with the concentrator by a tube (9). The tube (9) is connected directly or closed on one end by a semipermeable membrane (not shown). This arrangement facilitates the collection by stopping migration of large molecules according to the properties of the membrane used. This arrangement simplifies the sample collection and provides means of connecting the concentrator on-line to other devices, such as, for example, capillary analyzers, chromatography, PCR devices, enzymatic reactors, and the like. The tube (9) can also be used to supply a countercurrent flow of the leading electrolyte in an arrangement without a gel containing leading electrolyte.

In general, the gel for the leading electrolyte stabilization is formed by any uncharged material such as, for example agarose, polyacrylamide, pullulans, and the like. In some devices, the top surface is left open, or in some devices the top surface is closed, depending on the nature of the separation to be performed. If closed, the material used to cover the device is preferably a heat conducting, insulating material so as to prevent evaporation during the operation of an epitachophoresis device.

In general, the ring (circular) electrode is preferentially a gold-plated or platinum-plated stainless steel ring as this allows for maximum chemical resistance and electric field uniformity. Alternatively stainless steel and graphite electrodes may be used in some devices, particularly for disposable devices. Also, the ring (circular) electrode can be substituted with other moieties that provide similar function, e.g., by an array of wire electrodes. Moreover, a 2 dimensional array of regularly spaced electrodes may additionally or alternatively be used in epitachophoresis devices. An array of regularly spaced electrodes in a circular orientation may also be used in epitachophoresis devices. Furthermore, other electrode configurations may also be used to effect different electric field shapes based on the desired sample separation (e.g., for directing the focused zones). Such configurations are described as polygon arrangements of electrodes. When divided into electrically separated segments, a switched electric field is created for time dependent shape of the driving electric field. Such an arrangement facilitates sample collection in some devices.

### Epitachophoresis Device Operation

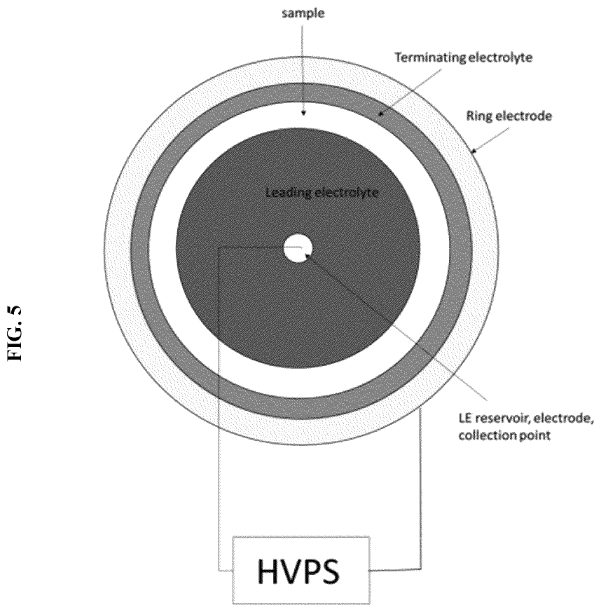

Epitachophoresis devices, such as those of the designs presented in FIG. 1-FIG. 4, are operated in either a two electrolyte reservoir arrangement, with the leading electrolyte followed by sample mixed with terminating electrolyte or with the sample mixed with the leading electrolyte followed by the terminating electrolyte, or in a three electrolyte reservoirs arrangement, as is presented in FIG. 5. In such an arrangement, the sample may be mixed with any conducting solution. Alternatively, when the sample contains suitable terminating ions the terminating electrolyte zone can be eliminated. Referring to FIG. 2A-FIG. 2B, upon filling the terminating electrolyte reservoir (2) with a mixture of sample and suitable terminating electrolyte and turning on the electric power supply (6), the ions start moving towards the center electrode (5) and form zones at the boundary between leading and terminating electrolytes (7). The concentrations of the sample zones during the migration adjust according to general isotachophoretic principles [Foret, F., Krivankova, L., Bocek, P., Capillary Zone Electrophoresis. Electrophoresis Library, (Editor Radola, B. J.) VCH, Verlagsgessellschaft, Weinheim, 1993.]. Thus, the low concentrated sample ions are concentrated and highly concentrated ones are diluted. Once the sample zone enters the electrolyte reservoir (4) the separation process is stopped, and the focused material is collected in the center of the device. In practice, final concentrations of migrating zones have a concentration comparable to that of the leading ion. Typically, concentration factors of anywhere from 2 to 1000 or even more are achieved using epitachophoresis.

In a three electrolyte reservoir arrangement, the sample is applied in between the leading and terminating electrolytes (see, for example, FIG. 5), and such an arrangement results in slightly faster sample concentration and separation as compared to a two electrolyte reservoir arrangement.

To avoid mixing, the leading electrolyte and the trailing electrolyte are stabilized by a neutral (uncharged) viscous media, e.g., agarose gel (see, for example, FIG. 2A-FIG. 2B, 3, which represents the leading electrolyte optionally contained within a gel or hydrodynamically separated from the terminating electrolyte).

All common electrolytes known to those skilled in the art that are used for isotachophoresis can be used with the present epitachophoresis devices when the leading ions have a higher effective electrophoretic mobility than that of the sample ion(s) of interest. The opposite is true for the selected terminating ions.

The device is operated either in positive mode (separation/concentration of cationic species) or in negative mode (separation/concentration of anionic species). The most common leading electrolytes for anionic separation using epitachophoresis include, for example, chloride, sulfate, or formate, buffered to desired pH with a suitable base, e.g., histidine, TRIS, creatinine, and the like. Concentrations of the leading electrolyte for epitachophoresis for anionic separation range from 5 mM-1 M with respect to the leading ion. Terminating ions then often include IVIES, MOPS, HEPES, TAPS, acetate, glutamate and other anions of weak acids and low mobility anions. Concentrations of the terminating electrolyte for epitachophoresis in positive mode range from: 5 mM-10 M with respect to the terminating ion.

For cationic separation common leading ions for epitachophoresis include, for example: potassium, ammonium or sodium with acetate or formate being the most common buffering counterions. Reaction hydroxonium ion moving boundary then serves as a universal terminating electrolyte formed by any weak acid.

In both positive and negative modes, the increase of the concentration of the leading ion results in proportional increase of the sample zone at the expense of increased electric current (power) for a given applied voltage. Typical concentrations are in the 10-100 mM range; however, higher concentrations are also possible.

Furthermore, in cases where only zone electrophoretic separation is sufficient, the device can be operated with only one background electrolyte.

Current and/or voltage programming is suitable for adjusting the migration velocity of the sample. It should be noted that in this concentric arrangement, the cross section area changes during the migration and the velocity of the zone movement is not constant in time. Thus, this arrangement does not strictly follow the isotachophoretic principle where the zones migrate with constant velocities. According to the mode of operation of the electric power supply (6) three basic cases may be distinguished: 1. Separation at Constant Current; 2. Separation at Constant Voltage; and 3. Separation at Constant Power.

Variables for the equations described below are as follows: d=distance migrated (d<0; r >); E=electric field strength; H=Electrolyte (gel) height; I=electric current; J=electric current density; κ=electrolyte conductivity; r=radius; S=cross-section area (area between the two electrolytes); u=electrophoretic mobility; v=velocity; and X=length from the center electrode to epitachophoresis boundary.

In the common mode of operation that uses constant electric current supplied by a high voltage power supply (HVPS), the migrating zone is accelerated as it moves closer to the center due to increasing current density. With regard to separation at constant current and using a device comprising a circular architecture, e.g., a device comprising one or more circular electrodes, the relative velocity at a distance, d, depends only on the mobility (conductivity) of the leading electrolyte, as is demonstrated by the derivation of the epitachophoresis boundary velocity at v at the distance d from the start radius r as follows:

### General Equations:

$$U = IR or E = J / κ ( Ohms ' s Law )$$

$$E = U / X ( electric field strength )$$

$$J = E κ ⇒ I = \frac{S U κ}{X} ; R = X / κ S$$

$$v = uE$$

$$S = 2 π X H$$

Epitachophoresis Boundary Velocity v at the distance d from the start with radius r:

v(d)=u_{L}I/2π(r−d)hκ_{L}=Constant/(r−d)

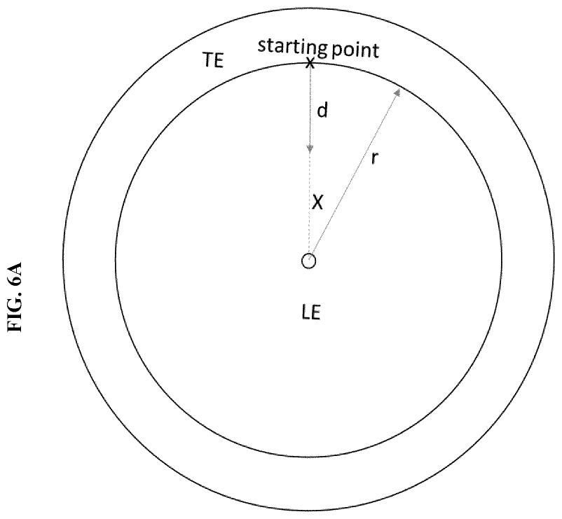

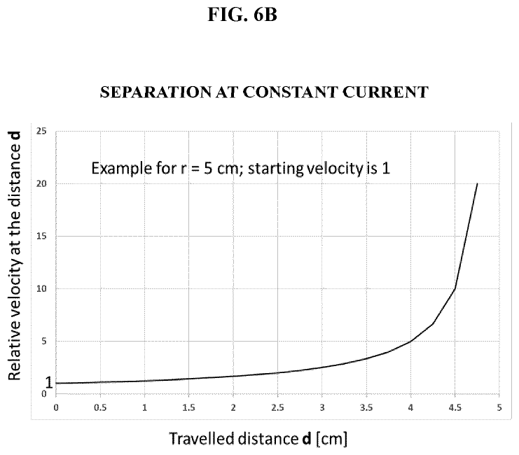

For a plot of the relationship of the distance traveled (d) vs. the relative velocity at the distance d at constant current, see FIG. 6B.

The ETP device may also be operated at constant voltage or constant power. The velocity of the electromigration also accelerates during the analyses performed at constant voltage and constant power.

### Epitachophoresis Using Exemplary Devices

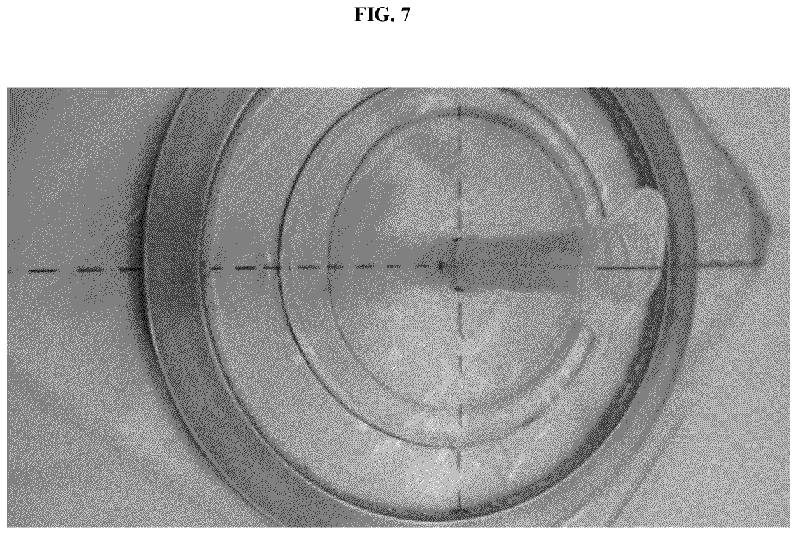

An epitachophoresis device, as presented in FIG. 7, was used to perform an epitachophoresis separation that focused sulfanilic acid dye (SPADNS) into a concentric ring. 1 W constant power was applied to effect epitachophoresis in the epitachophoresis device.

Referring to FIG. 7, SPADNS was focused into a concentric ring-shaped focused zone, which can be seen as the red zone of FIG. 7. The upper half of the red circle showed that the height of the zone was approximately 5 mm. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

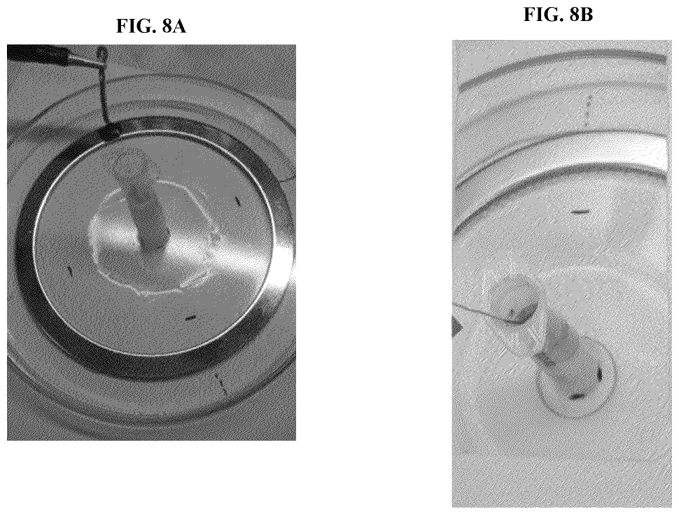

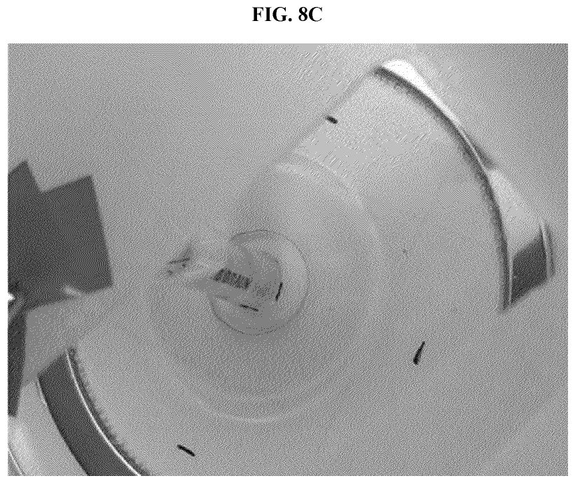

An epitachophoresis device (FIG. 8A) was used to perform epitachophoresis to focus sulfanilic acid (SPADNS). The device of FIG. 8A had a circular architecture and a circular gold electrode with a diameter of 10.2 cm. HCl-histidine (pH 6.25) was used as the leading electrolyte and was contained in 10 mL of an 0.3% agarose gel which had a diameter of 5.8 cm. 15 mL of MES Tris (pH 8.00) was used as the trailing electrolyte. The syringe reservoir of the device contained the leading electrolyte HCl His (pH 6.25). 300 μl of SPADNS at a concentration of 0.137 mM was prepared in trailing electrolyte and loaded into the device. To effect epitachophoresis, a constant power of 1 W was used.

Referring to FIG. 8B, SPADNS was focused into a concentric ring-shaped focused zone, which can be seen as the red zone of FIG. 8B. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

Furthermore, the epitachophoresis device of FIG. 8A was used to perform epitachophoresis to focus a 30 nt oligomer (ROX-oligo). The device of FIG. 8A had a circular architecture and a circular gold electrode with a diameter of 10.2 cm. 10 mM HCl-histidine (pH 6.25) was used as the leading electrolyte was contained in 10 mL of an 0.3% agarose gel which had a diameter of 5.8 cm. 15 mL of 10 mM MES Tris (pH 8.00) was used as the trailing electrolyte. The syringe reservoir of the device contained the leading electrolyte HCl His (pH 6.25) at a concentration of 100 mM. 75 μl of ROX-oligo at a concentration of 100 μM was prepared in trailing electrolyte and loaded into the device. To effect epitachophoresis, a constant power of 1 W was used.

Referring to FIG. 8C, ROX-oligo was focused into a concentric ring-shaped focused zone, which can be seen as the blue zone of FIG. 8C. As the epitachophoresis zone moved from the edge towards the center of the device, eventually the focused zone of the ROX-oligo entered the center of the device and was collected in the center of the device, thereby demonstrating focusing and recovery of a desired sample using epitachophoresis.

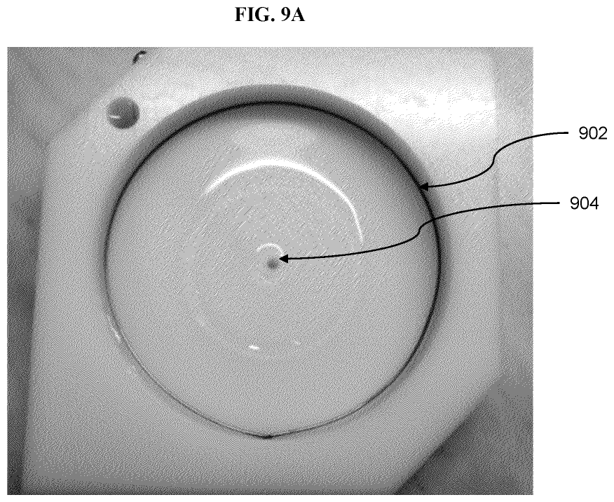

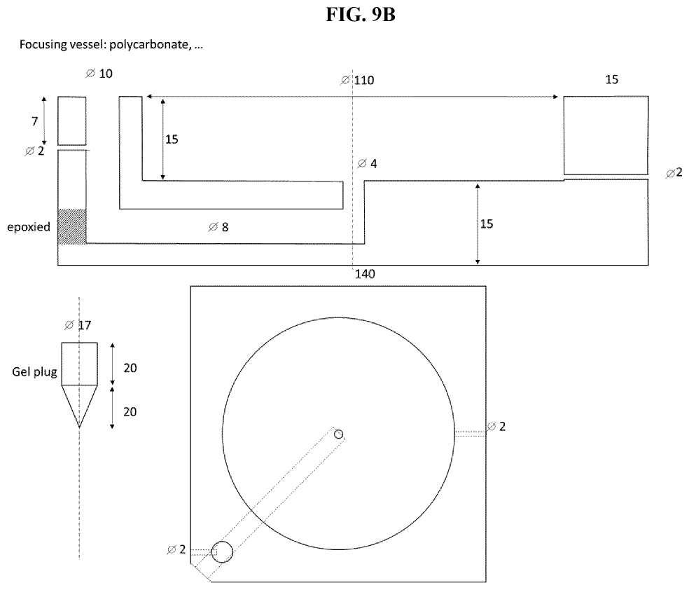

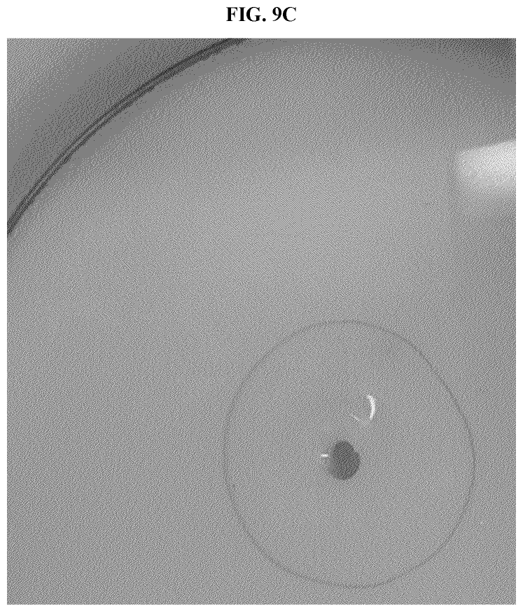

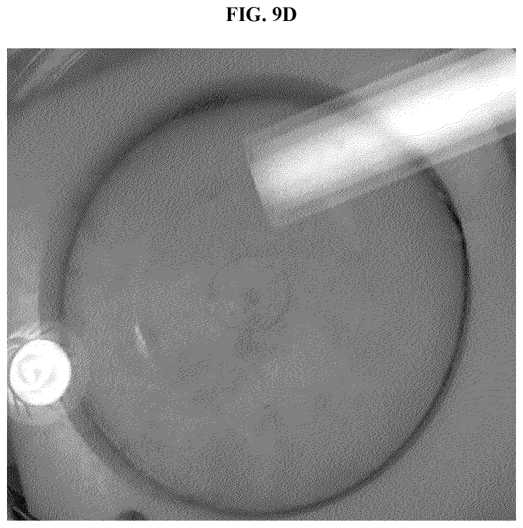

An epitachophoresis device (FIG. 9A-FIG. 9B) was used to perform epitachophoresis to focus sulfanilic acid dye (SPADNS), which was subsequently collected from said device (FIG. 9C-FIG. 9D). The device of FIG. 9A-FIG. 9B had a circular architecture and a circular stainless steel wire electrode (902) with a diameter of 11.0 cm. Circular stainless steel wire electrode 902 marks the outside of a circular channel where epitachophoresis takes place. Electrode reservoir or sample collection reservoir (904) is in the center of the circular channel. Referring to FIG. 9B, the numbers of the schematic represent dimensions in millimeters. 20 mM HCl-histidine (pH 6.20) was used as the leading electrolyte. Either 5 mL of 10 mM IVIES Tris (pH 8.00) was used as trailing electrolyte with an 0.3% agarose gel in LE, wherein the gel had a diameter of 8.9 cm (FIG. 9C) and was formed prior to introduction of TE, or 15 mL of 10 mM MES Tris (pH 8.00) was used as trailing electrolyte contained in an 0.3% gel which had a diameter of 5.8 cm (FIG. 9D) and was formed prior to introduction of TE. The electrode reservoir of the device contained leading electrolyte HCl His (pH 6.25) at a concentration of 100 mM.

Referring to FIG. 9C, 150 μl of SPADNS at a concentration of 0.137 mM was prepared in 15 mL of trailing electrolyte and loaded into the device. To effect epitachophoresis, a constant power of 2 W was used. SPADNS was focused into a concentric ring-shaped focused zone, which can be seen as the red zone of FIG. 9C. As the epitachophoresis zone moved from the edge towards the center of the device, eventually the focused zone of the SPADNS entered the center of the device and was collected in the center of the device, thereby demonstrating focusing and recovery of a desired sample using epitachophoresis. The recovered SPADNS had a 40-fold absorbance increase as compared to the absorbance of the initial 15 mL SPADNS-containing sample.

Referring to FIG. 9D, 150 μl of SPADNS at a concentration of 0.137 mM was prepared in 15 mL of trailing electrolyte and loaded into the device. To effect epitachophoresis, a constant power of 2 W was used. SPADNS was focused into a concentric ring-shaped focused zone, which can be seen as the red zone of FIG. 9D. As the epitachophoresis zone moved from the edge towards the center of the device, eventually the focused zone of the SPADNS entered the center of the device and was collected in the center of the device, thereby demonstrating focusing and recovery of a desired sample using epitachophoresis. The recovered SPADNS had a 40-fold absorbance increase as compared to the absorbance of the initial 15 mL SPADNS-containing sample.

The epitachophoresis device of FIG. 9A-FIG. 9B was also used to perform epitachophoresis to focus SPADNS from a physiological saline solution in a device that did not use a gel. 20 mM HCl-histidine (pH 6.20) was used as the leading electrolyte. 13 mL of 10 mM IVIES Tris (pH 8.00) was used as trailing electrolyte, which was further mixed with 3 mL of 0.9% NaCl. The electrode reservoir of the device contained leading electrolyte HCl Histidine (pH 6.25) at a concentration of 100 mM.

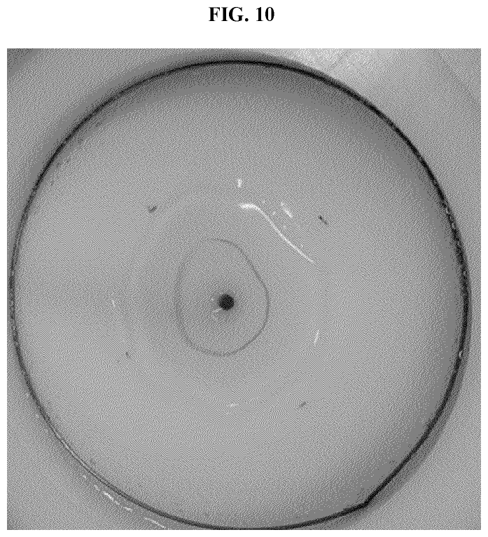

Referring to FIG. 10, 150 μl of SPADNS at a concentration of 0.137 mM was prepared in 13 mL of trailing electrolyte mixed with 3 mL of 0.9% NaCl and loaded into the device. To effect epitachophoresis, a constant power of 2 W was used. SPADNS was focused into a concentric ring-shaped focused zone, which can be seen as the red zone of FIG. 10. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

The epitachophoresis device of FIG. 9A-FIG. 9B was also used to perform epitachophoresis to separate and to focus SPADNS and Patent Blue dye with acetic acid as a spacer. 20 mM HCl-histidine (pH 6.20) was used as the leading electrolyte. 5 mL of 10 mM IVIES Tris (pH 8.00) was used as trailing electrolyte, which was further mixed with 150 μl of 10 mm acetic acid, 150 μl of 0.1 mM Patent Blue dye, and 150 μl of 0.137 mM SPADNS. The effective mobility values (10⁻⁹ m²/Vs) of SPADNS, acetic acid, and Patent Blue dye were 55, 42, 7, and 32, respectively. The electrode reservoir of the device contained leading electrolyte HCl His (pH 6.25) at a concentration of 100 mM. No gel was used in the channel of this device for this experiment, however gel was present on the top of the device platform.

Referring to FIG. 11, the mixture of trailing electrolyte, SPADNs, acetic acid, and Patent Blue dye was loaded into the device. To effect epitachophoresis, a constant power of 2 W was used. SPADNS was focused into a concentric ring-shaped focused zone, which can be seen as the red zone/inner zone of FIG. 11, and Patent Blue dye was focused into a concentric ring-shaped focused zone as well, which can be seen as the blue zone/outer zone of FIG. 11. As the epitachophoresis zones moved from the edge towards the center of the device, eventually the focused zones of the SPADNS and the Patent Blue dye entered the center of the device sequentially and may be collected separately in the center of the device, thereby demonstrating separation, focusing and recovery of a desired samples using epitachophoresis.

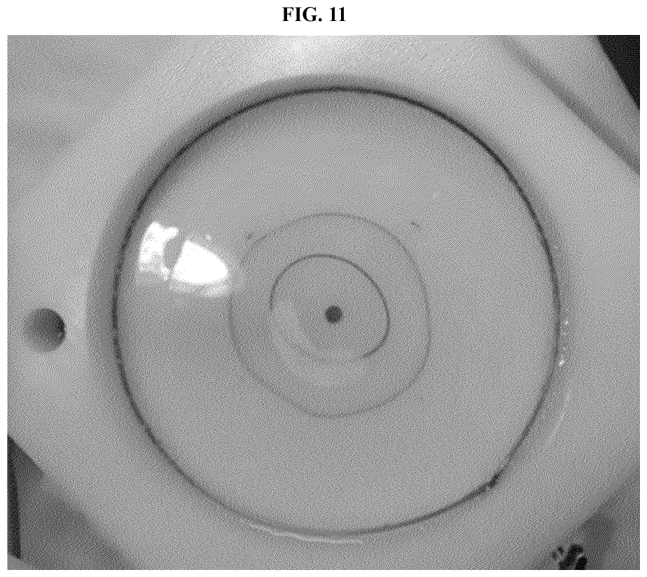

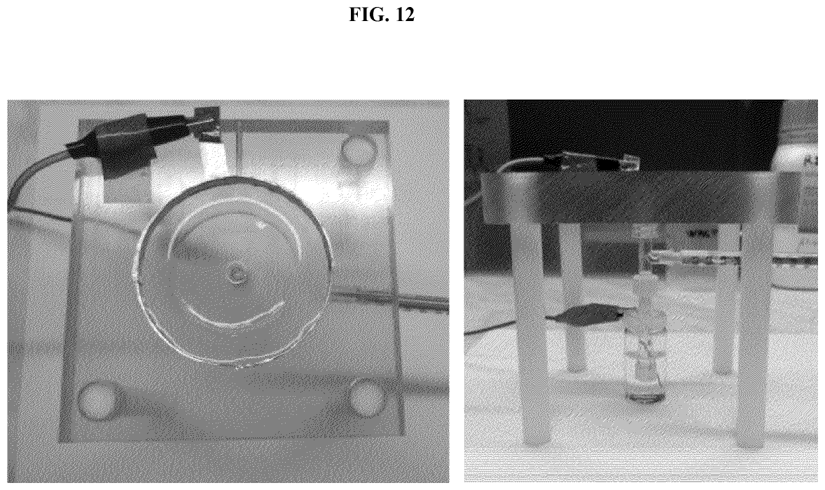

An epitachophoresis device was designed for effecting epitachophoresis (FIG. 12). The device of FIG. 12 had a circular architecture and a circular copper tape electrode with a diameter of 5.8 cm.

System for Effecting Epitachophoresis with Conductivity-Based Sample Detection

Device Construction

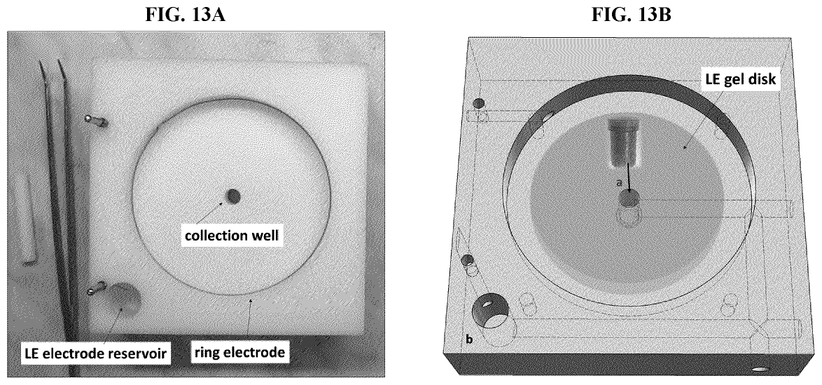

In accordance with previous examples, an epitachophoresis device with a large sample volume capacity was machined with a circular separation channel. FIG. 13A and FIG. 13B show two views of the structure of the device. The wire ring electrode (1 mm diameter stainless steel wire; radius of 55 mm) was attached on the edge of the circular separation compartment. The sample volume was defined by the space between the ring electrode and an agarose stabilized leading electrolyte disk (radius of 35 mm). Thus, the applicable sample volume was 5.7 ml for every mm of its height. The second electrode was placed in the leading electrolyte reservoir on the side of the device. The ring electrode was connected to the upper banana type connector shown in FIG. 13A. The bottom banana connector was attached to a 3 cm long, 0.4 mm diameter platinum wire electrode positioned in the leading electrode reservoir (“b” in the scheme shown in FIG. 13B). To prevent possible interferences from electrolysis products, migrating out from the leading electrolyte reservoir towards the center collection well (“a” in the scheme shown in FIG. 13B), a 9 mm ID internal channel with a total length of 20 cm long was drilled inside the device. The side openings after drilling of the device were plugged by silicon septa. The central collection well with the diameter of 9 mm was drilled through the device and closed from the bottom by a moving Ertacetal® rod sealed by a rubber o-ring.

For every analysis, a plastic vial with a semipermeable membrane (Slide-A-Lyzer™ MINI Dialysis Units 2000 Da MWCO, Thermo Fisher Scientific, USA) was inserted into the central collection well after filling it with the leading electrolyte all the way to the leading electrode reservoir. To minimize the volume, the Slide-A-Lyser was cut in half by a razor blade creating a collection cup with a volume of less than 200 microliters. Next a 0.3% agarose gel disk (70 mm diameter, 4 mm thick) with a central 8 mm hole was prepared in the leading electrolyte, positioned in the center of the device and covered by a 75×1 mm round glass plate also having a center 8 mm hole to avoid bubble accumulation. Although various electrophoretic separation modes may be applied (e.g., zone electrophoresis, isoelectric focusing, or displacement electrophoresis), we have used epitachophoresis with an electrolyte system comprising leading (LE) and terminating (TE) electrolytes. The sample solution in the terminating electrolyte was applied by a syringe into the space between the gel disk and ring electrode. The polarity of the electric current connection was selected so that anionic sample components migrated from the ring electrode towards the collection well in the device center. After the focused sample zone entered the collection cup, the electric current was turned off, and the sample was pipetted out for further use. The empty collection cup was lifted up by the moving rod and discarded.

Electrodriven Separation Conditions

Separations were performed in negative mode, where ion served as leading ion (effective mobility 79.1×10⁻⁹ m² V⁻¹ s⁻¹). Leading electrolyte (LE) contained 100 mM HCl-Histidine buffer at pH 6.2 and terminating electrolyte (TE) contained 10 mM TAPS titrated by TRIS to pH 8.30. The agarose stabilized leading electrolyte disk was prepared in 20 mM leading electrolyte (HCl-Histidine; pH 6.25). All buffers were prepared in deionized water. The power supply was provided by a PowerPac 3000 (BioRad), which was run at constant power mode at 2 W (this corresponds approximately to 16 mA and 120 V at the beginning of the analysis). Analysis took approximately 1 hour 10 mA and 200 V at the end of analysis).

Sample Detection

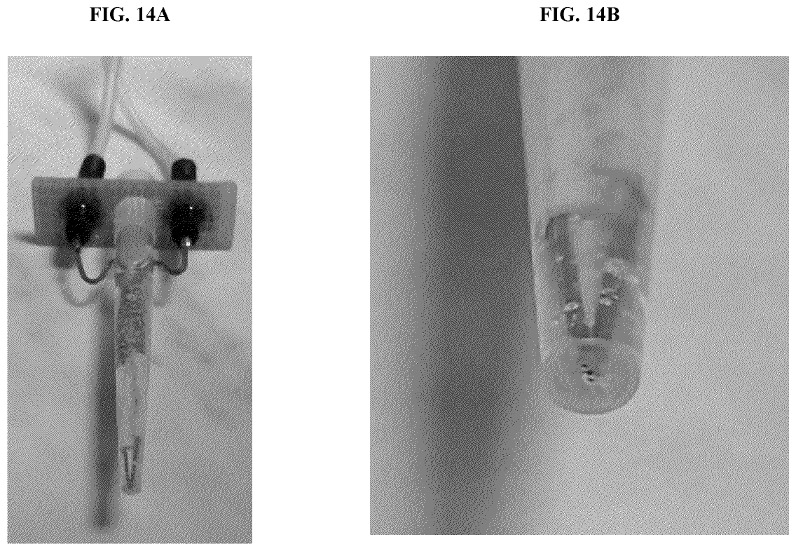

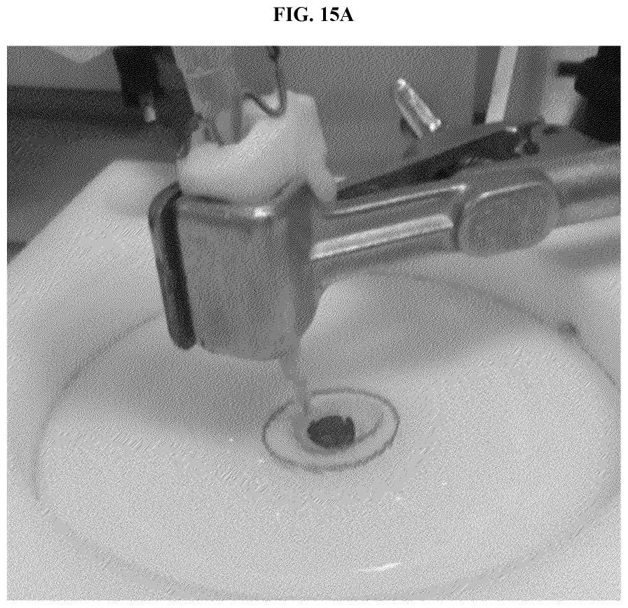

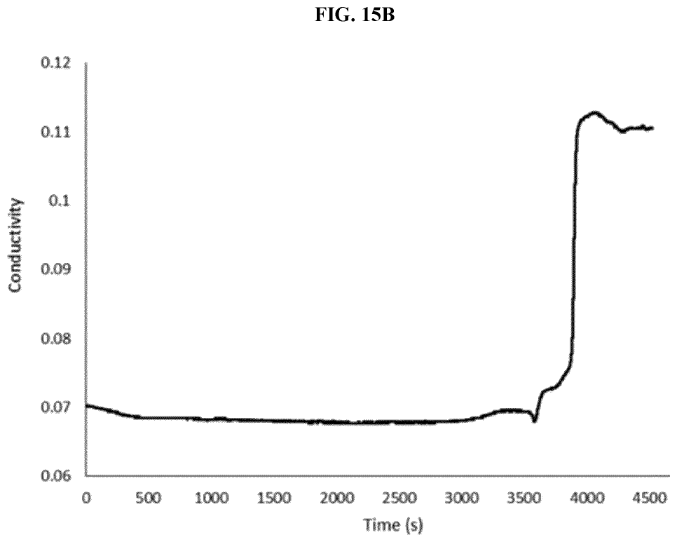

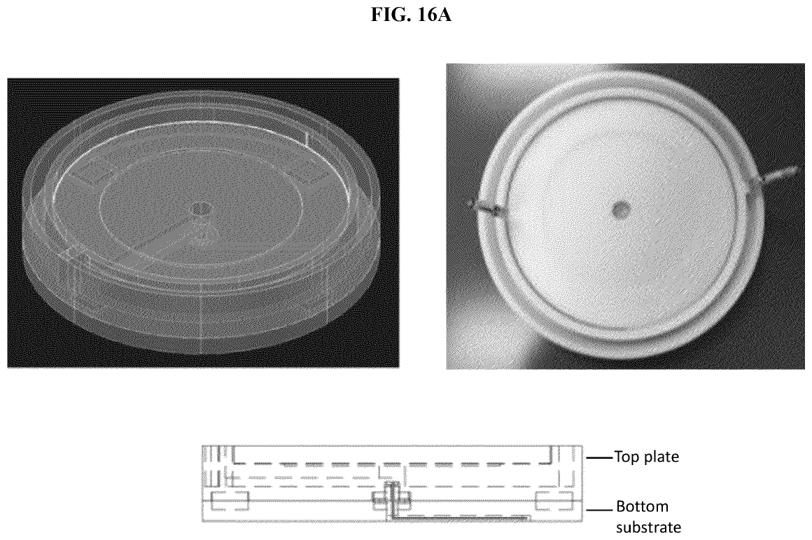

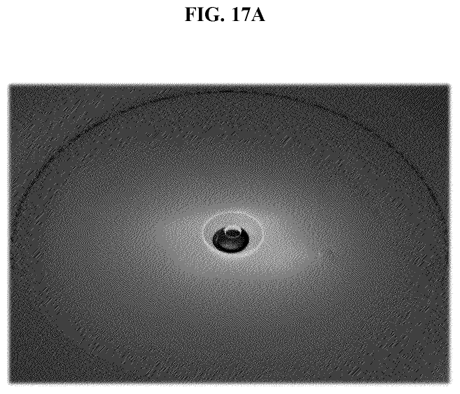

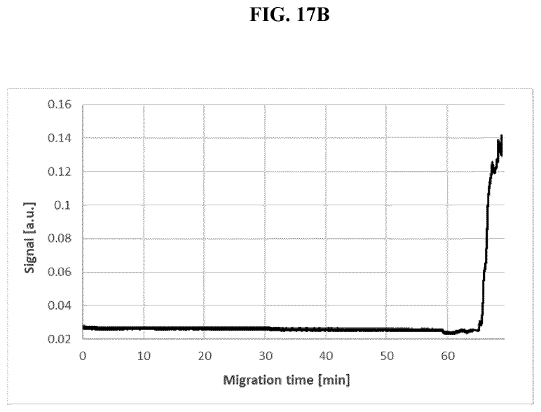

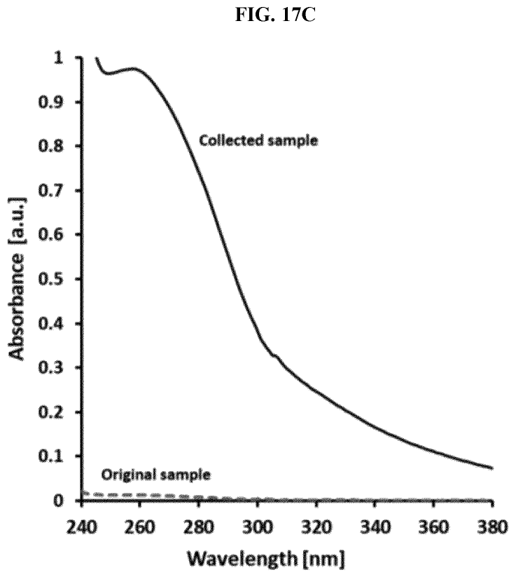

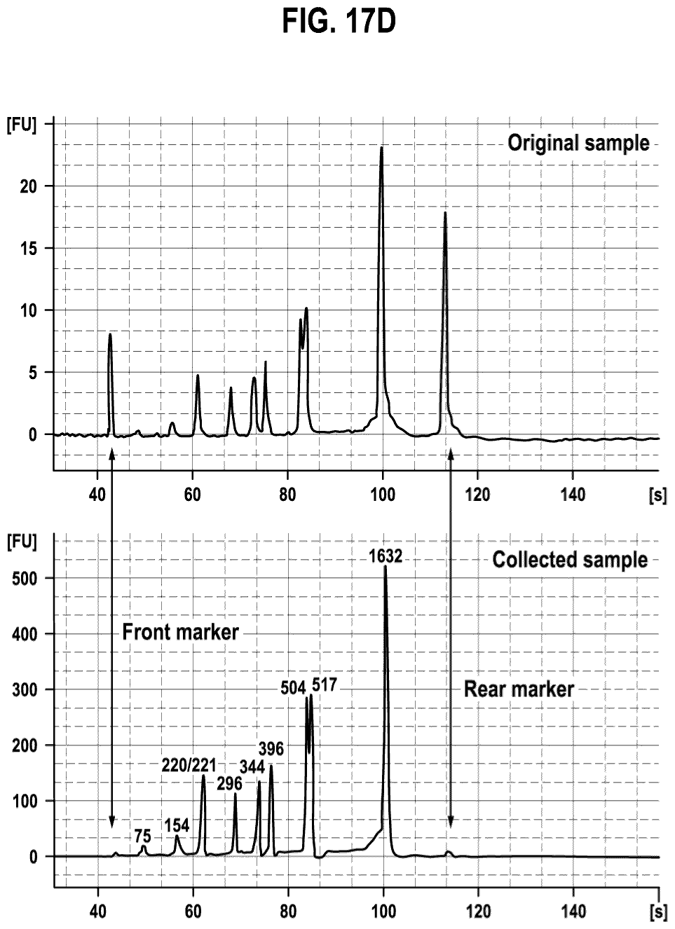

For detecting the samples, a surface resistivity detection cell was constructed and connected to the conductivity detector of a commercial ITP instrument (Villa Labeco, Sp.N.Ves, Slovakia). The detection cell was prepared as follows: two platinum (Pt) wires (300 μm×2 cm long) were attached to connectors matching the ITP instrument. The opposite ends of the Pt wires were inserted into a 1 mL pipette tip, which was then filled by a quick setting epoxy resin. Finally, 1 mm of the pipette tip with the epoxy wires embedded inside was cut by a razor blade exposing flat epoxy surface with two round Pt electrodes. See FIG. 14A and FIG. 14B. The detection cell was mounted in a laboratory stand gently touching the surface of the agarose gel disk close to the collection vial, as exemplified in FIG. 15A. This system for detection was employed to generate the conductivity trace of FIG. 15B and FIG. 17B.

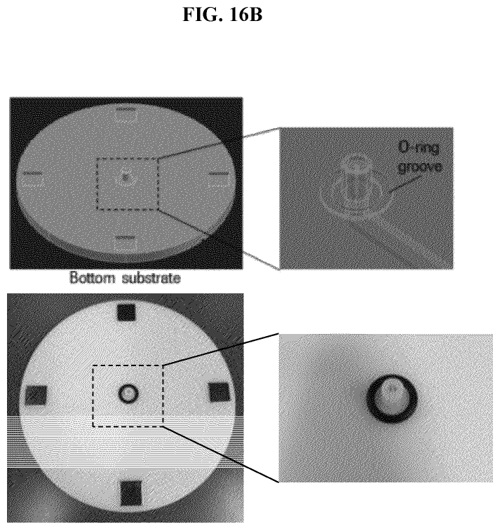

Another exemplary system was also constructed for sample detection during epitachophoresis. In this system, surface resistivity detection probes consisting of two platinum (Pt) wires with a diameter of 500 μm were incorporated within the bottom substrate (i.e., bottom plate) of an epitachophoresis device, as shown in FIG. 16A and FIG. 16B. The tips of the wires were within proximity of the semipermeable membrane from the bottom via dedicated channels within the central pillar on the bottom substrate. The opposite ends of the wires were connected to the conductivity detector of a commercial ITP instrument (Villa Labeco, Sp.N.Ves, Slovakia). The top plate, serving as the epitachophoresis device, was assembled with the bottom substrate using magnets, while the o-ring (see FIG. 16B) enabled complete sealing between the two substrates in order to prevent any leakage.

Chemicals

Buffer components: L-histidine monohydrochloride monohydrate (99%), L-histidine (99%), N-tris(hydroxymethyl)methyl-3-aminopropanesulfonic acid (TAPS; 99.5%) and tris-(hydroxymethyl)aminomethane (TRIS; 99.8%) were purchased from Sigma-Aldrich (USA). Agarose NEED ultra-quality Roti®garose with low electroendosmosis was purchased from Carl Roth (Germany). Acetic acid and anionic dye Patent blue V sodium salt were from Sigma-Aldrich; red anionic dye SPADNS (1,8-dihydroxy-2-(4-sulfophenylazo)naphthalene-3,6-disulfonic acid trisodium salt was from Lachema, Brno, Czech Republic.

Focusing of SPADNS and Patent Blue

To test the above-described exemplary device comprising epitachophoresis and electrical sample detection, the device was used to focus and detect test analytes: SPADNS and Patent Blue. Leading electrolyte (LE): HCl-HIS buffered to pH 6.2. Trailing electrolyte (TE): TAPS-TRIS buffered to pH 8.3. The gel was formed from 20 ml of polyacrylamide gel 6% in 20 mM LE. 100 mM LE was added to the electrode reservoir. Sample solution: 15 ml of 10 mM TE+150 μL 0.1 mM SPADNS+150 μL 0.1 mM Patent blue. The sample solution in the terminating electrolyte was applied by a syringe into the space between the gel disk and ring electrode. The device was run in constant power mode with P=2 W. FIG. 15A provides an image of the focusing of the SPADNS and Patent Blue, showing the conductivity sample detection near the sample collection well. FIG. 15B provides the conductivity trace for this sample focusing and shows a marked change in conductivity/resistivity that was due to the transition between LE and TE, which encompassed the focused zone of sample (SPADNS and Patent blue).

DNA Analysis

Low molecular weight dsDNA ladder labeled with Fluorescein (ten fragments from 75 base pairs—bp to 1622 bp) was from Bio-Rad, USA. The DNA concentration in the collected fraction was evaluated using Qubit fluorometer (Invitrogen, Carlsbad, CA, USA) by using the high sensitivity dsDNA Qubit quantitation assay kit. The concentration of the target molecule in the sample was reported by a fluorescent dye emitting only when bound to the DNA. The collected fractions were further analyzed using the chip CGE-LIF instrument Agilent 2100 Bioanalyzer (Agilent, Santa Clara, CA, United States). This analysis provided size information of the collected DNA fragments in the sample using the high sensitivity DNA reagent kit (Agilent, United States).

DNA Focusing

Electrophoretic mobilities of DNA fragments above 50 bp are approximately 37×10⁻⁹ m²/Vs in free solution, and short fragments (˜20-50 bp) may deviate by only ˜10%. Based on these mobilities, we designed a discontinuous electrolyte system suitable for focusing of all sample DNA fragments into a single concentrated zone. A discontinuous electrolyte system may include different gel structure (or presence of gel), pH value of the buffer, ionic strength of the buffer, and/or ions. For experimental testing, we selected a fluorescein labeled low molecular range DNA ladder with fragment sizes ranging from 75 to 1632 bp. The fluorescence of the sample with only one fluorophore per DNA fragment was illuminated by a 2 cm radius laser beam (FIG. 17A). The surface resistivity detection was used to indicate the transition of the LE/TE boundary close to the collection well (conductivity trace shown in FIG. 17B). The overall change in resistivity from LE to TE was used as an indicator of the sample location. Based on this change, the voltage was turned off and separation was stopped. The collected fraction was analyzed by both UV spectrometry (absorbance measurements) (FIG. 17C) and Bioanalyzer-based analysis (FIG. 17D). The lower signal intensity of the front and rear markers in FIG. 17D (added at the same amount to the initial sample and to the final sample according the manufacturer's instructions) was due to the higher DNA concentration in the collected fraction. In both cases the ˜30× concentration increase in the collected fraction corresponded to the decrease of the sample volume from the starting 15 mL to the sample collection volume of 280 μL in this exemplary embodiment. The volume of the migrating DNA zone prior to entering the collection cup was much smaller (˜3 μL) and the final fraction concentration depends mainly on the volume of the selected collection vial.

ETP with Voltage-Based Sample Detection

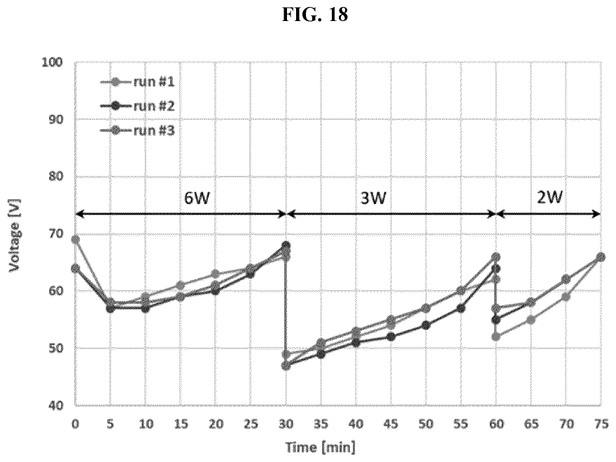

In the present example, three independent ETP runs were performed to focus and collect cfDNA from 1 mL of plasma using an ETP device, and the voltage was measured during the time course of each of the three independent ETP runs. Each of the three ETP runs was 75 minutes in length. The power level at the beginning of each ETP run was 6 W. The power level was subsequently lowered to 3 W at 30 min., and then lowered to 2 W at 60 min. The results obtained during each of the three independent ETP runs are presented in FIG. 18.

Referring now to FIG. 18, in each stage during which the power was kept constant, the voltage gradually increased. In the last stage (2 W power, 60 min. to 75 min.), the voltage was 65 V when the focused zone comprising the nucleic acid molecules migrated into the collection cup. Voltage profiles were observed to be consistent when comparing each of the three independent runs, which indicated that the voltage feedback from the power supply could be used to monitor the location of the nucleic acid molecules within the device.

ETP with Optical-Based Sample Detection

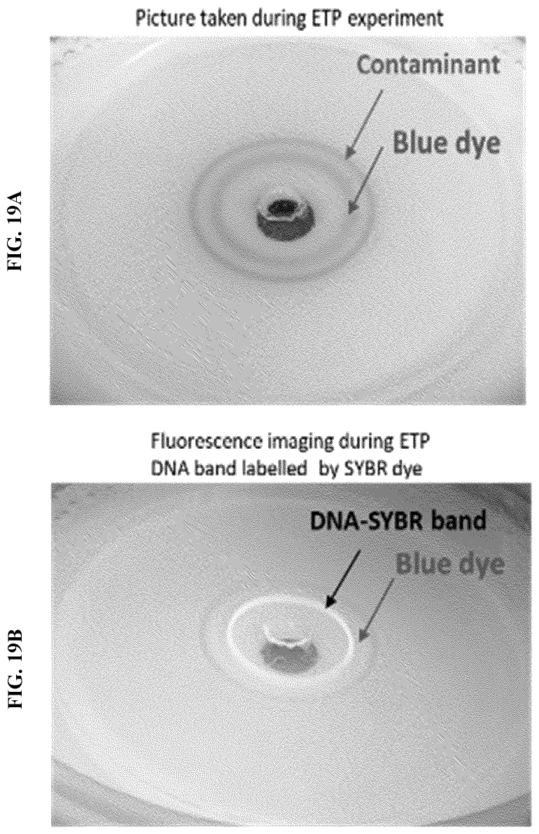

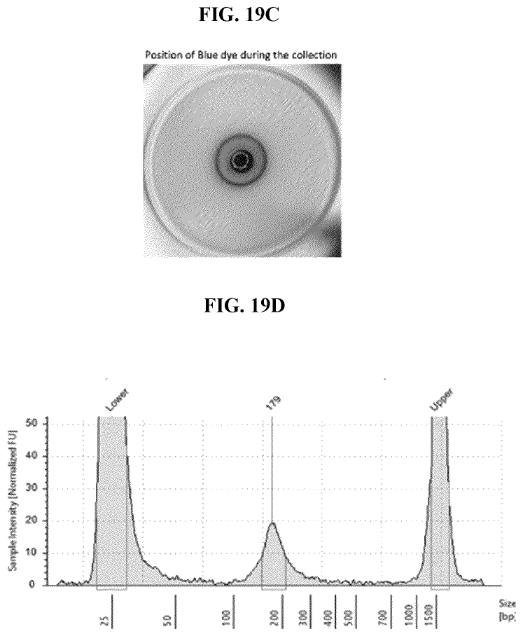

In the present example, ETP runs were performed using optical detection with colored dye to monitor the position of nucleic acids during the ETP runs. A chromatic dye with an electrophoretic mobility lower than nucleic acids can be used to enable optical tracking of the location of nucleic acids. For example, such dyes include Brilliant blue FCF, Indigo carmine, Sunset yellow FCF, Allura red, Fast green FCF, Patent blue V and Carmoisine. In the present example, two independent ETP runs were performed to focus and collect nucleic acid molecules using brilliant blue dye as an optical marker (see ETP Run 1: FIG. 19A-FIG. 19B; and ETP Run 2: FIG. 19C-FIG. 19D).

FIG. 19A presents an image of an ETP device during an ETP run in which Brilliant Blue dye was used as an optical marker during focusing and collection of nucleic acid molecules, and also in which SYBR-gold dye was further used to monitor the position of the nucleic acid molecules. The electrophoretic mobility of the blue dye was lower than that of the nucleic acids, and, as such, the blue dye migrated after the focused zone comprising the nucleic acid. Contaminant appeared as a brown-colored focused zone (see FIG. 19A). In addition to the photographic image of FIG. 19A, a fluorescence-based image was taken during the ETP run (see FIG. 19B). The fluorescence-based image of FIG. 19B demonstrates that the focused zone comprising the band comprising DNA labeled with SYBR-gold migrated faster than the brilliant blue dye.

FIG. 19C presents an image of an ETP device during an ETP run in which Brilliant Blue was used as an optical marker during focusing and collection of nucleic acid molecules. A plasma sample comprising cfDNA was used for the ETP run of FIG. 19C and FIG. 19D. The ETP run was stopped once the dye band reached the collection well. After focusing and collecting the cfDNA, analysis was performed on the focused and collected cfDNA sample using an Agilent TapeStation system (see FIG. 19D). The electropherogram (see FIG. 19D) showed a peak at 179 bp representing the desired cfDNA molecules which were focused and collected during the ETP run, thereby demonstrating the utility of Brilliant blue dye to monitor the location of nucleic acids.

ETP with Thermal-Based Sample Detection

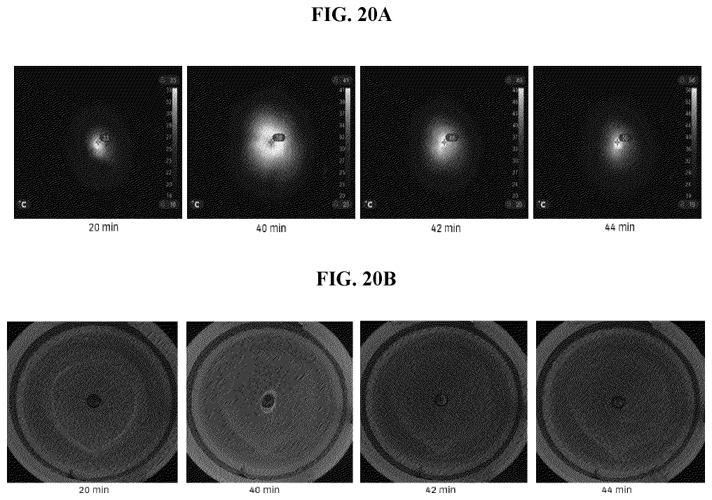

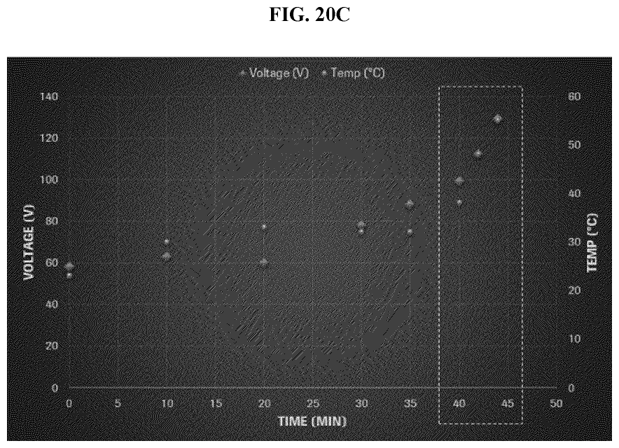

In the present example, an ETP run was performed in which thermal imaging was used during focusing and collection of a DNA ladder. For the thermal imaging of the present example, an infrared-based thermal imaging camera (SEEK thermal ShotPro) was used. Additionally, fluorescence imaging was used. Thermal and fluorescent images were taken at four sequential time points of 20 min., 40 min., 42 min., and 44 min. (see FIG. 20A-FIG. 20B). Furthermore, the voltage feedback from the power supply was measured during the ETP run.

FIG. 20A presents thermal images that were taken at four time points during the ETP run: 20 min., 40 min., 42 min., and 44 min. It was observed that the temperature at the center of the device increased by 17° C. (from 38° C. to 55° C.) between 40 and 44 min., during which the DNA ladder, visible as the green-fluorescent ring in FIG. 20B, moved into the center collection cup.

FIG. 20C presents the voltage and temperature change over time during the ETP run of the present example. It was observed that the trend of the voltage change over time was similar to that of the temperature change over time. As such, the voltage feedback from the power supply provided an additional means for monitoring the location of DNA ladder molecules within the device.

### Example 1: Cell Free Nucleic Acid Isolation/Purification by ETP

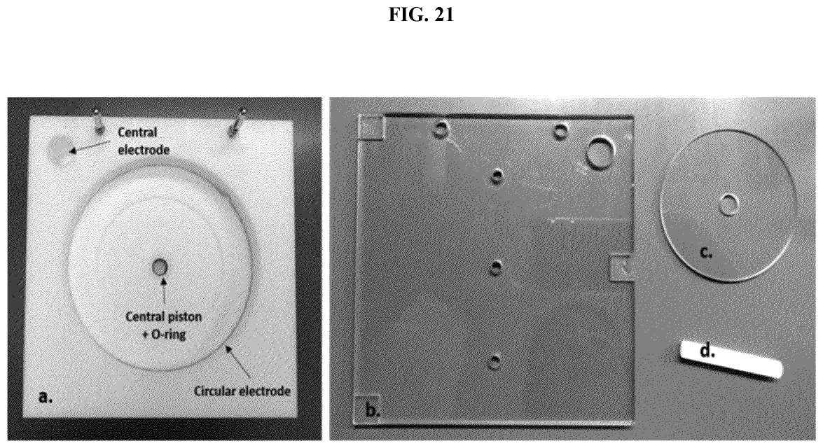

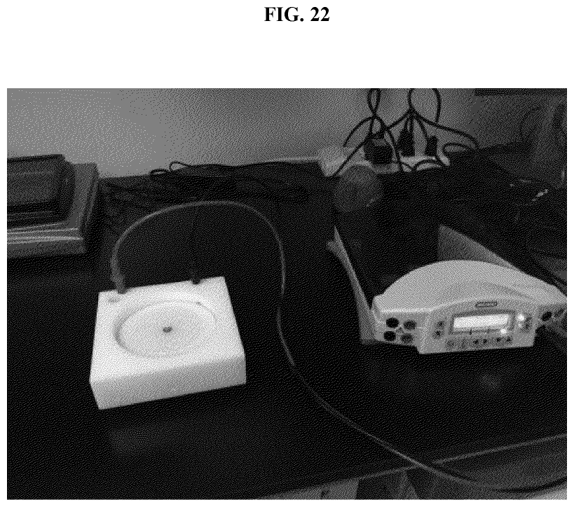

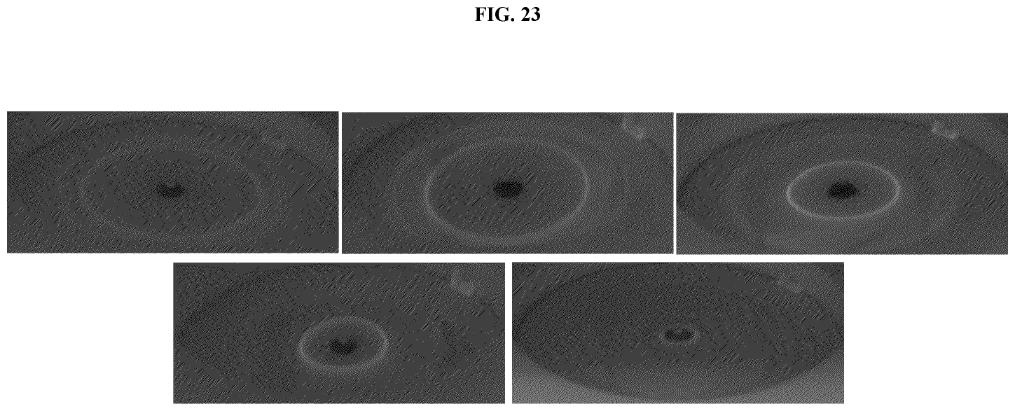

An epitachophoresis device and experimental setup (see FIGS. 21-23) were used to perform ETP to effect isolation (purification) of cell free nucleic acids comprising cell free DNA. The device had a circular architecture and a circular electrode (see FIGS. 21 and 23).

Prior to setting up and performing ETP to isolate/purify cfDNA, ETP buffers, the agarose gel of the ETP device, the shortened dialysis unit, and sample(s) of proteinase K digested plasma were prepared. The leading electrolyte (LE) buffer, which comprised HCl-Histidine pH 6.25, was prepared, and the trailing electrolyte (TE) buffer, which comprised TAPS-Tris pH 8.30, was prepared. The agarose gel used with the ETP device was prepared by mixing an amount of agarose appropriate for a desired agarose percentage gel with LE buffer in an Erlenmeyer flask.

Proteinase K digested plasma was prepared by first thawing a plasma sample at room temperature, after which the sample was mixed and a desired volume withdrawn and dispensed into a nuclease free tube. Next, Proteinase K was added, and the solution was mixed well then incubated at 37-70° C., depending on the sample.

ETP-based isolation/purification of cfDNA generally proceeded as follows. The ETP system was prepared by first moving the mobile central piston (see FIG. 21) to a lower position by using a Teflon stick (see FIG. 21) or a pair of tweezers. The central electrode channel was filled with LE buffer (25 mL of LE+ 1.25 μL SYBR Gold (if used for visualization of DNA band)) via the corner opening of the ETP platform, and filling was stopped when the central opening was completely filled. A dialysis unit was secured in the central opening via the O-ring (see FIG. 21). The dialysis unit was then filled with LE buffer (and SYBR gold solution if desired). The agarose gel that was prepared as described above was carefully transferred from the mold to the ETP device and secured. The circular cover was then placed on the gel.

A sample mixture, generally containing 15 mL TE buffer+1 μL SYBR Gold (if used for visualization of the DNA band)+50 bp DNA Ladder (if using as a marker)+PK pretreated plasma sample, was prepared and pipetted into the gap between the gel and the circular electrode of the ETP device. Finally, the secondary lid was placed on top of the device.

The power supply was then prepared by plugging the ETP device into the power supply. The power supply was set at a constant power of between 1 to 8 W (dependent on the amount of plasma sample used), and ETP was effected for approximately 1-2 hrs. by turning on the power supply, which ETP focused the one or more target analytes into one or more focused zones (one or more ETP bands).

In some instances, sample was monitored and collected as follows. A blue light source and the appropriate filter were used to monitor the movement of the DNA focused zone (ETP band) if SYBR gold was included in the ETP run. Once the DNA was collected in the dialysis unit, the power was switched off. If size selection of DNA/analytes was desired, only the DNA focused zone or zones (ETP band or bands) of interest were collected, as further described below. Once the power was switched off, the LE buffer was removed from the corner opening of the ETP device, and then the TE buffer and gel were removed from the device. Next, the sample contained in the dialysis unit was collected. The mobile central piston was then moved to an upper position. In some instances, the collected DNA solution was subsequently cleaned by using a mixture of KAPA Pure beads and eluted in 30-50 μL of a TRIS.HCl solution.

In some instances, analysis of the collected cfDNA was performed by using a Qubit fluorimeter (Invitrogen, Carlsbad, CA, USA) by using the high sensitivity dsDNA Qubit quantitation assay kit. The concentration of the target molecule in the sample was reported by a fluorescent dye emitting only when bound to the DNA. In some instances, analysis of the collected cfDNA was performed by using the chip CGE-LIF instrument Agilent 2100 Bioanalyzer (Agilent, Santa Clara, CA, United States). This analysis provided size information of the collected DNA fragments in the sample using the high sensitivity DNA reagent kit (Agilent, United States).

### Example 2: ETP-Based Isolation/Purification of cfDNA

In the present example, cfDNA was isolated/purified by ETP-based isolation/purification as generally described in Example 1 with the following modifications. DNA ladder was added to the plasma sample, and SYBR gold was used for visualization of the DNA zone. The sample comprised 1 mL of plasma comprising cfDNA and 200 ng of a DNA ladder.

Referring now to FIG. 23, time lapse photos were taken of ETP-based isolation/purification of DNA from a 1 mL plasma sample, which isolated/purified cfDNA and DNA ladder. SYBR gold was used for visualization of the DNA zone (see FIG. 23).

### Example 3: ETP-Based Isolation/Purification of cfDNA

In the present example, cfDNA was isolated and collected from a 1 mL plasma sample by ETP-based isolation/purification as generally described in Example 1, with the following modifications. SYBR gold was not used. Following ETP-based/purification and subsequent collection of cfDNA, either a single KAPA pure beads based clean up step (1× SPRI bead) or two KAPA pure based bead clean up steps (2× SPRI bead) were performed. cfDNA was also isolated/purified from plasma samples (1 mL) by using a spin column method in which an AVENIO kit was used, followed by a single KAPA pure beads-based clean up step; and by using a “UNA method” which comprised removal of genomic DNA contamination using a two bead-based cleanup, i.e., two cleanup steps, each using the same type of bead, followed by the spin column method using the AVENIO kit. Following isolation/purification and cleanup of cfDNA using the methods described above, the concentration of cfDNA was measured using QUBIT-based analysis, and the concentration of cfDNA in ng was recorded.

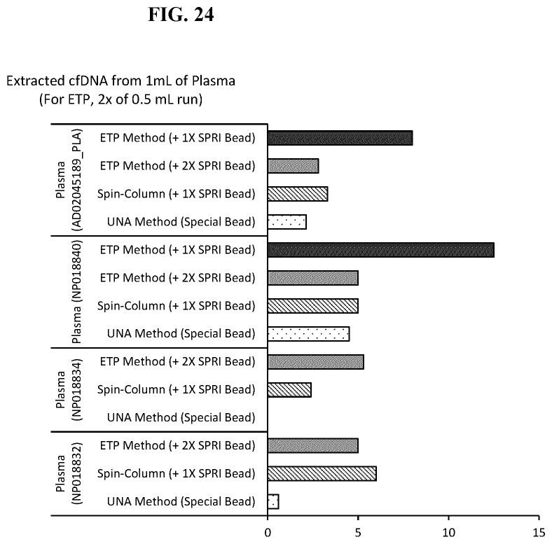

Referring now to FIG. 24, the results demonstrated the extraction of cfDNA by using the ETP-based isolation and collection comprising either one or two bead clean up steps. Isolation and collection of cfDNA by ETP-based isolation and collection yielded about 2.5 times the amount of cfDNA as compared to the other methods used in the present example. It was noted that performing ETP-based isolation and collection of cfDNA followed by one bead-based cleanup step comprised a higher amount of cfDNA as compared to ETP-based isolation and collection of cfDNA that comprised two bead-based clean up steps.

### Example 4: ETP-Based Isolation/Purification of cfDNA

In the present example, DNA which comprised cfDNA and a DNA ladder was isolated/purified from 1 mL of plasma by ETP-based isolation/purification as generally described in Example 1, with the following modifications. 1 mL of plasma was spiked with 60 ng of DNA ladder. Following isolation/purification and sequent collection of the cfDNA and DNA ladder, a Bioanalzyer run as generally described in Example 1 was used to perform size-based analysis of the isolated/purified and collected DNA sample.

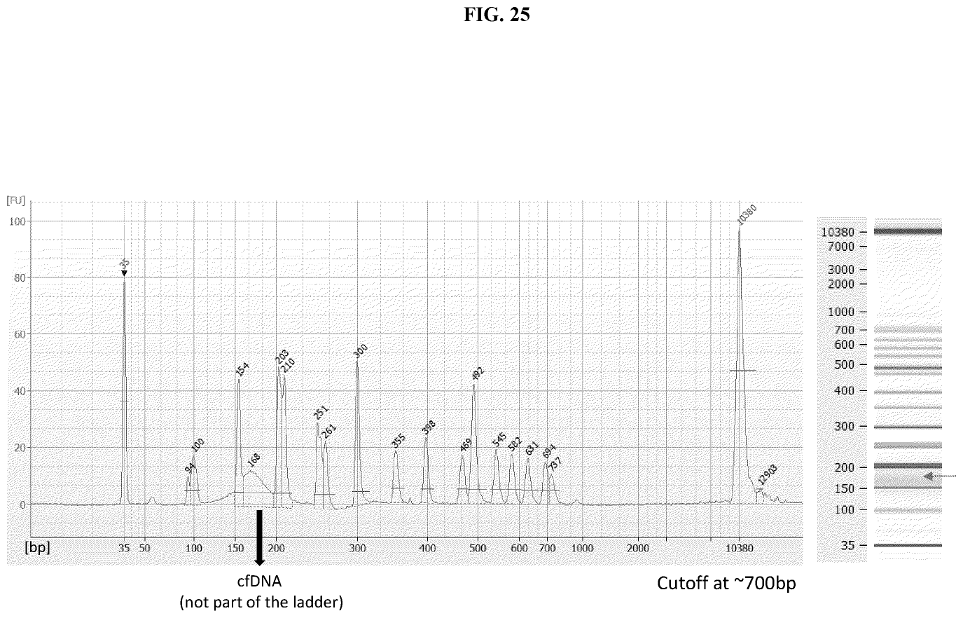

Referring now to FIG. 25, the results demonstrated the presence of cfDNA in the isolated/purified and collected sample as evidenced by the appearance of fluorescent signal and band which did not correspond to that of the DNA ladder as cfDNA appeared as a band from about 150 to about 200 bp in length (see FIG. 25).

### Example 5: ETP-Based Isolation/Purification of cfDNA

In the present example, cfDNA was isolated/purified from 1 mL of plasma sample by ETP-based isolation/purification as generally described in Example 1, with the following modifications. In the present example, no SYBR gold or DNA ladder were used. Following isolation/purification and subsequent collection of the cfDNA, a Bioanalzyer run as generally described in Example 1 was used to perform size-based analysis of the isolated/purified and collected DNA sample.

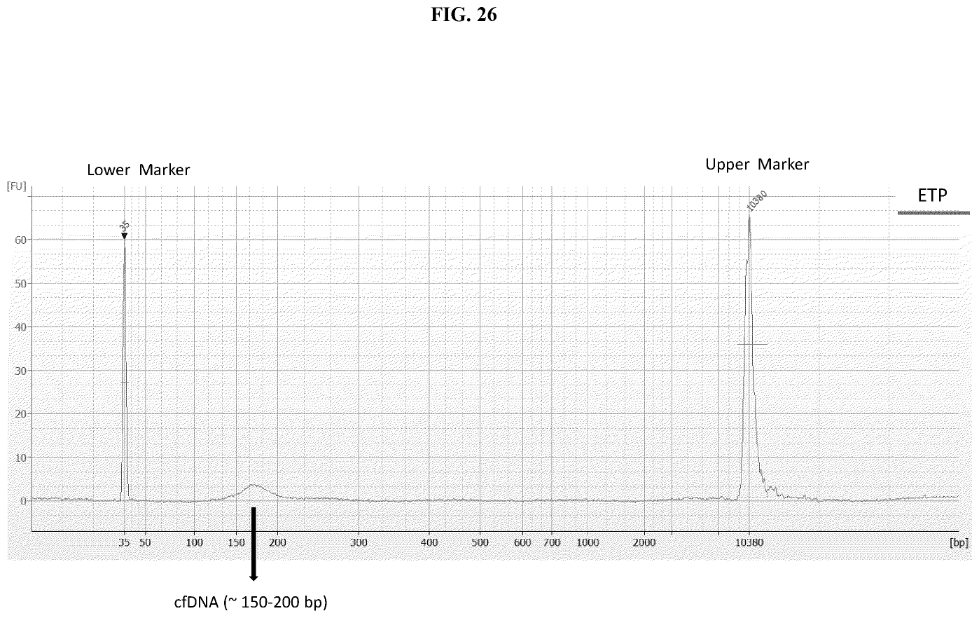

Referring now to FIG. 26, the results demonstrated the presence of cfDNA in the isolated/purified and collected sample as evidenced by the appearance of fluorescent signal and band from about 150 to about 200 bp in length (see FIG. 26). It is noted that markers of 25 bp and 10,300 bp were used as standards.

### Example 6: ETP-Based Isolation/Purification of cfDNA

In the present example, cfDNA was isolated/purified from 1 mL of plasma sample by ETP-based isolation/purification as generally described in Example 1, with the following modifications. In the present example, cfDNA was isolated/purified and subsequently collected from 1 mL of a plasma sample by varying the buffer concentration the percentage of the gel, and the stoppage time of the ETP run, such that various different size ranges of DNA were isolated/purified and subsequently collected, which allowed for the enhanced isolation/purification of cfDNA from fragmented genomic DNA.

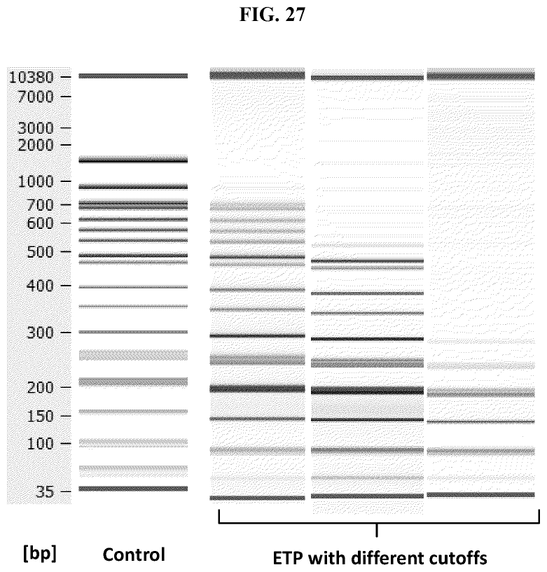

Referring now to FIG. 27, the results of the ETP-based isolation/purification and subsequent collection with varied buffer concentration, gel percentages, and stoppage times are presented. Electrophoretic-based analysis of the results of the ETP runs demonstrated that various DNA size cutoffs were achieved by ETP-based isolation/purification and subsequent collection (see FIG. 27).

### Example 7: ETP-Based Isolation/Purification of Circulating Tumor DNA

In the present example, circulating tumor DNA was isolated/purified and subsequently collected from a 1 mL plasma sample by ETP-based isolation/purification as generally described in Example 1, with the following modifications. In addition to the ETP-based isolation/purification, a spin column-based method using an AVENIO kit was used to isolate ctDNA from a 4 mL plasma sample in a separate assay. Also, following the ETP-based isolation/purification and subsequent collection of the ctDNA, a KAPA pure bead-based cleanup step was performed. Additionally, QUBIT-based analysis of the isolated/purified and subsequently collected ctDNA was performed as generally described in Example 1.

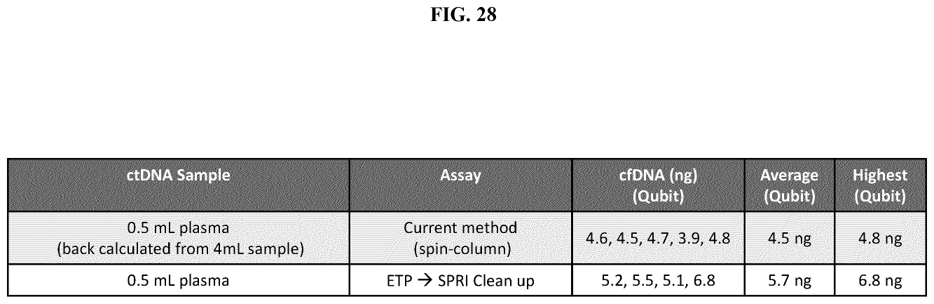

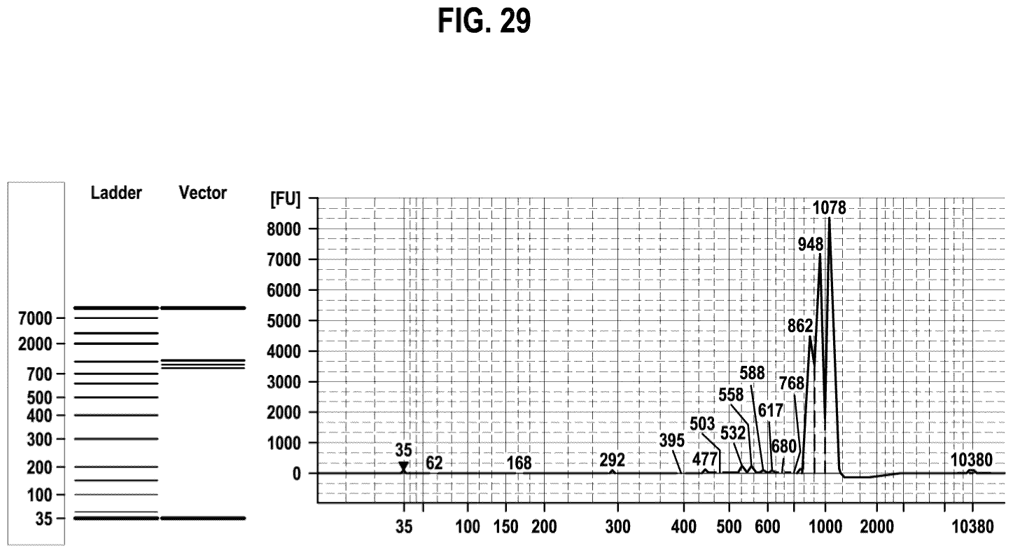

Referring now to FIG. 28, the results of the ETP-based isolation/purification and subsequent collection of ctDNA from 0.5 mL of plasma are presented. It is noted that the results for the spin column-based method were back calculated from a 4 mL sample to 0.5 mL. Five readings were taken of each sample. The results presented in FIG. 29 demonstrate that the yield of ctDNA isolated/purified and subsequently collected by the ETP-based method exceeded that of the spin column-based method. The yield of ctDNA by ETP-based method averaged about 5.7 ng, with a highest yield of 6.8 ng, and ranged from about 5.1 ng to about 6.8 ng.

### ETP-Based Isolation/Purification Using an ETP Upper Marker

In the present example, an ETP upper marker to be used during ETP-based isolation/purification was generated.

One approach for generating a labeled upper marker to be used during ETP is to digest a plasmid at one restriction site, and then to subsequently generate an amplicon of a desired size, e.g., a 1003 bp amplicon, using the appropriate primers to generate an amplicon of 1003 bp in size. Optionally, the amplicon can be fluorescently labeled.

Alternatively, a labeled upper marker was generated as follows. A vector was cut at three different restriction sites using three different restriction enzymes to generate fragments of 744 bp, 875 bp, and 1067 bp. Following digestion, the digested product was cleaned up, and subsequently the three pieces of vector were each fluorescently labeled. After cleanup, an Agilent Bioanalyzer was used to analyze the ETP upper marker (see FIG. 29), which confirmed generation of the upper marker with three pieces of 744 bp, 875 bp, and 1067 bp.

Such an ETP upper marker can be used during ETP-based methods and with ETP-based devices to indicate a cutoff point at which collection of a target analyte, e.g., DNA, can be stopped. For example, fluorescently labeled, or otherwise detectably labeled, ETP upper maker can be generated of such a size that it is larger than the target analyte to be collected during the ETP based method. By monitoring the marker throughout the ETP run, the user or automated machine is able to stop the run before the marker falls into a collection tube, thereby allowing target analyte smaller than the marker to be captured while larger contaminating analytes are left out as they are positioned behind the upper marker. Moreover, the ETP upper marker itself is not collected, and as such can be used at high quantity and with various detectable labels since it will not interfere with downstream assays. In particular, an ETP upper marker can be used in cfDNA isolation/purification methods as it can aid in the exclusion of genomic DNA. For example, ETP upper marker can be about 1000 bp in some instances.

ETP-Based Isolation of Target Analytes from Urine

In the present example, ETP-based isolation and/or purification is used to isolate/purify one or more target analytes from a urine sample.

Urine samples generally comprise various different target analytes, such as, for example, nucleic acids, cfNAs, biomarkers, proteins, and/or extracellular vesicles. ETP-based isolation and/or purification can be used to isolate and/or purify any one or more of these target analytes using one or more ETP runs as follows. A urine sample of any volume, generally of a volume from 1 mL to about 50 mL, is provided, which urine sample comprises one or more target analytes. The urine sample is concentrated and buffer exchanged by using a centrifugation-based method comprising use of AMICON® Ultra Centrifugal filters. As a result of the buffer exchange and concentration procedure, an initial enrichment of the one or more target analytes, such as cfNAs, proteins, and/or extracellular vesicles, e.g., urinary exosomes, is accomplished, and a desalting of the urine sample is accomplished.

In some instances, prior to the centrifugation-based concentration and buffer exchange, a filtration step, such as a vacuum filtration step, can be performed to remove unwanted materials, such as, for example, unwanted cells and/or unwanted cellular debris. Such a filtration step can be accomplished by using centrifugation-based filtration and/or vacuum-based filtration using a filter size selected to remove the unwanted materials, such as a 0.22 um filter membrane, to remove said unwanted materials, including solid debris in urine. The filter may have a size of 0.10 to 0.20 μm, 0.20 to 0.25 μm, 0.25 to 0.35 μm, 0.35 to 0.45 μm, 0.45 to 0.50 μm, 0.50 to 1 μm, 1 to 10 μm, 10 to 25 μm, 25 to 30 μm, 30 to 45 μm, 45 to 55 μm, or greater than 55 μm.

Following the optional filtration step, the centrifugation-based concentration and buffer exchange is performed. The sample is concentrated by centrifugation, such as by using an AMICON® Ultra Centrifugal Filter with a 3K-10K molecular weight cutoff (MWCO). Following concentration, a desired buffer, such as a wash buffer, is added to the concentrated sample, mixed (sometimes referred to as a “buffer exchange” if the added buffer is different than the buffer prior to centrifugation), and a further centrifugation is performed. This step in which a wash and/or buffer exchange is performed can be repeated as many times as desired.

The centrifugation may be a filter for a molecular weight cutoff of 1,000 to 2,000, 2,000 to 3,000, 3,000 to 4,000, 4,000 to 5,000, 5,000 to 6,000, 6,000 to 7,000, 7,000 to 8,000, 8,000 to 9,000, 9,000 to 10,000, 10,000 to 15,000, or 15,000 to 20,000. In some embodiments, material under the MWCO may be used in the ETP device. In other embodiments, material above the MWCO may be used. The centrifuge may be run at a force of 3 to 100×g, 100 to 200×g, 200 to 500×g, 500 to 1,000×g, 1,000 to 3,600×g, 3,600 to 5,000×g, or more than 5,000×g. The radius of rotation of centrifugation may be from 50 to 100 mm, 100 to 200 mm, 200 to 300 mm, 300 to 400 mm, 400 to 500 mm, or more than 500 mm. The revolutions per minute (RPM) may be from 200 to 1,000, 1,000 to 5,000, 5,000 to 10,000, 10,000 to 15,000, 15,000 to 20,000, 20,000 to 25,000, or more than 20,000. The centrifuge may spin for 1 to 5 min, 5 to 10 min, 10 to 15 min, 15 to 20 min, 20 to 25 min, 25 to 30 min, 30 to 60 min, or greater than 60 min.

In some instances, buffer exchange can be accomplished through a dialysis-based method, if so desired, and this buffer-exchanged sample can then be concentrated by a centrifugation-based method as described above.

In some instances, following sample concentration and buffer exchange, the sample is used for ETP-based isolation/purification of one or more biomarkers, such as, for example, one or more nucleic acids and/or one or more proteins. ETP-based isolation/purification of such nucleic acids can occur by ETP-based isolation/purification procedures described in the present examples, including examples that discuss isolation/purification of cell free nucleic acids from plasma samples.

In some instances, following sample concentration and buffer exchange, a lysis and/or protein digestion step can be performed prior to introduction of the sample into the ETP device. Such steps may be effected at least in part based on the origin of target nucleic acids and/or the desired target nucleic acids to collect. Origins of nucleic acids found in urine include, for example, epithelial cells shed from the urinary tract, freely circulating cfDNA, and exosomes.

If total nucleic acid recovery is desired, wherein all nucleic acids regardless of their origin, are to be isolated/purified, a lysis step and/or protein digestion step can be performed using appropriate reagents. For example, proteinase K may be used to effect protein digestion. Moreover, such lysis and protein digestion steps can allow for all nucleic acids to be released from, for example, cells, exosomes, and histones (in case of cfDNA) prior to ETP-based isolation/purification.

If nucleic acid recovery which does not include nucleic acids that originated from epithelial cells shed from the urinary tract is desired, a centrifugation step is performed to effect removal of these epithelial cells prior to lysis and/or protein digestion steps. Following the centrifugation, the supernatant is collected, while a pellet, which is presumed to contain the epithelial cells, is discarded. The target nucleic acids that can be subsequently isolated/purified by ETP include cfDNA as well as nucleic acids encapsulated in exosomes.

If nucleic acid recovery that targets cfDNA is desired, a protein digestion step, such as with proteinase K, is performed prior to introducing the sample into an ETP device. No lysis step is performed, and furthermore any sources of unintentional lysis are avoided to prevent the release of nucleic acids from cells that may be present in the urine sample.

Following any of the lysis and/or protein degradation steps discussed above, the sample can be loaded onto an ETP device and ETP-based isolation/purification performed to isolate/purify the one or more target analytes, e.g., nucleic acids, by ETP-based isolation/purification procedures described in the present examples, such as Example 8, which discusses isolation/purification of cell free nucleic acids from plasma samples. As discussed throughout the instant disclosure, any one or more of the conditions in which ETP is performed, such as, for example, buffer conditions, agarose gel percentage, etc., can be optimized to allow for isolation/purification of target analytes from a mixture of analytes, such as isolation/purification of target nucleic acids from a mixture of nucleic acids.

### Urine Pretreatment for ETP

A urine sample may be obtained. The urine sample may be voided urine. Bacteria and any solid debris may be removed by filter sterilization with a 0.25 μm to 45 μm filter. The urine sample may be spun in a centrifuge for 20 minutes at 3000×g using AMICON® Ultra Centrifugal filters with a 3 k to 10 k molecular weight cutoff. Buffer exchange may be performed with 10 mM Tris HCl pH 8 or 10 mM terminating electrolyte buffer. A 10 ml volume of buffer may be added to the to AMICON® tube, and the tube may be spun for additional 20 min or until volume of concentrate is 1 ml.

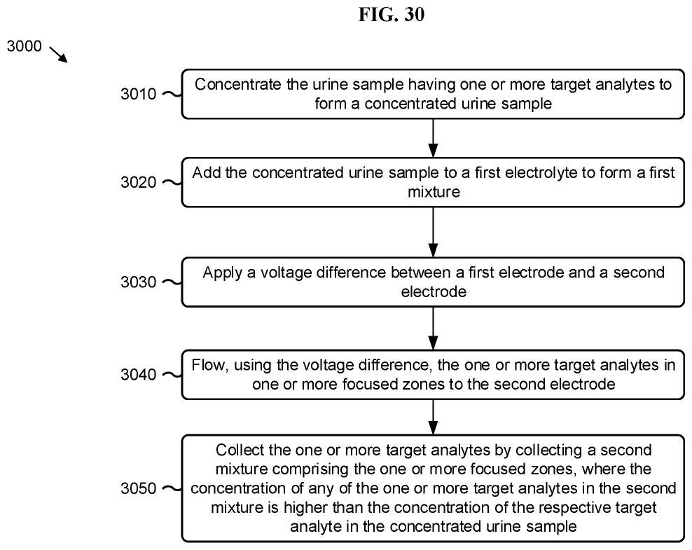

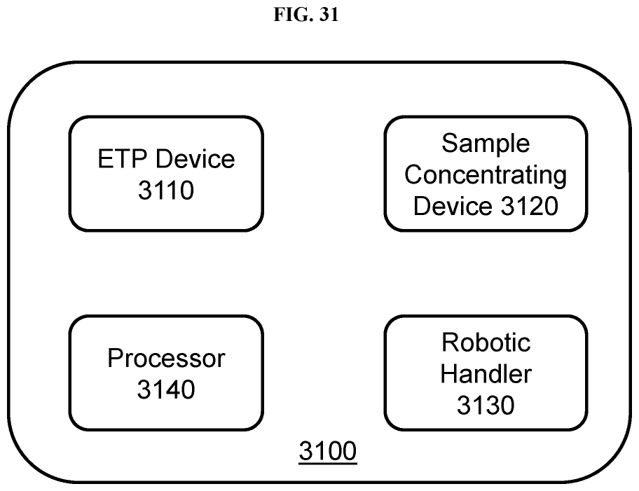

FIG. 30 is a flowchart of an example process 3000 associated with devices and methods for urine sample analysis. In some implementations, one or more process blocks of FIG. 30 may be performed by a system (e.g., system 3100). In some implementations, one or more process blocks of FIG. 30 may be performed by another device or a group of devices separate from or including the system. Additionally, or alternatively, one or more process blocks of FIG. 30 may be performed by one or more components of system 3100 of FIG. 31, such as ETP device 3110, sample concentrating device 3120, robotic handler 3130, and/or processor 3140.

At block 3010, process 3000 may include concentrating the urine sample to form a concentrated urine sample. The concentrated urine sample may have a concentration of the one or more target analytes that is at least 10 times higher than an initial concentration of the one or more target analytes in the urine sample. In some embodiments, the concentration in the concentrated urine sample may be 20 to 30 times, 30 to 40 times, 40 to 50 times, or 50 times or more higher than the initial concentration. The one or more target analytes comprise DNA, RNA, or a combination thereof. In addition or alternatively, the one or more target analytes may include cell-free nucleic acids, circulating tumor nucleic acids, biomarkers, proteins, extracellular vesicles, or a combination thereof. The one or more target analytes may include any analytes described herein.

Concentrating the urine sample may include one or more of vacuum filtration, desalting, buffer exchange, extracellular vesicle enrichment, exosome enrichment, cell lysis, protein degradation, or centrifugation-based cell-removal. Concentrating the urine sample may include buffer exchange, and the pH of the concentrated urine sample is in a range from 6.0 to 8.5, including about 8.0. The buffer may be Tris HCl or may be any terminating electrolyte buffer described herein. The buffer is an ETP-compatible buffer.

Concentrating the urine sample may include centrifuging the urine sample. Centrifuging the urine sample comprises centrifuging at a force in a range from 1,000 to 3,600×g or at any force described herein.

Concentrating the urine sample may include filtering the urine sample by molecular weight. Filtering the urine sample by molecular weight may include removing components of the urine sample with a molecular weight above or below a cutoff in a range of 3,000 to 10,000 or any cutoff described herein.

At block 3020, process 3000 may include adding the concentrated urine sample to a first electrolyte to form a first mixture. The first electrolyte may be the terminating electrolyte.

At block 3030, process 3000 may include applying a voltage difference between a first electrode and a second electrode. The first electrode may be disposed in the first mixture. The second electrode may be disposed in a second electrolyte. The second electrolyte may be the leading electrolyte. The first electrolyte may be different from the second electrolyte. The second electrolyte may be contained in a gel. The second electrolyte may be hydrodynamically separated from the first electrolyte. The first electrolyte and the second electrolyte may be separated by a membrane. The first electrode and the second electrode may be any electrodes with an ETP device described herein.

At block 3040, process 3000 may include flowing, using the voltage difference, the one or more target analytes in one or more focused zones within the second electrolyte to the second electrode. The focused zones may be sections where the target analytes are concentrated within the first electrolyte or the second electrolyte. The target analytes in a particular focused zone may include ions with the same or similar mobility in an applied electric field. The one or more focused zones may be any such focused zones or ETP bands described herein. Each focused zone may include a separate target analyte. Process 3000 may also include flowing the one or more target analytes in one or more focused zones in the first mixture (or the first electrolyte) before flowing the one or more target analytes in the one or more focused zones within the second electrolyte. The zones may exit the area with the first electrolyte and enter the area with the second electrolyte. The focused zones may number 1, 2, 3, 4, 5, or more than 5.

At block 3050, process 3000 may include collecting the one or more target analytes by collecting a second mixture comprising the one or more focused zones. The concentration of any of the one or more target analytes in the second mixture is higher than the concentration of the respective target analyte in the concentrated urine sample. The concentration of any of the one or more target analytes in the second mixture is 2 to 5 times, 5 to 10 times, 10 to 50 times, or more than 50 times higher than the concentration of the respective target analyte in the concentrated urine sample. As examples, the initial urine sample may be 10 to 50 ml, including 10 to 20 ml, 20 to 30 ml, 30 to 40 ml, or 40 to 50 ml. As examples, the volume of the concentrated urine sample may be 0.5 ml to 1.0 ml, 1.0 to 1.5 ml, 1.5 to 2.0 ml, or 2.0 to 2.5 ml. Process 3000 may increase throughput rates of obtaining analytes from a unconcentrated urine sample by at least 50% or at least a factor of 2, 3, 4, 5, 6, 7, 8, 9, or 10 over conventional techniques (e.g., columns).

Process 300 may include performing any in vitro diagnostic assay or method described herein on any or all of the one or more target analytes in the second mixture.

Process 3000 may include additional implementations, such as any single implementation or any combination of implementations described and/or in connection with one or more other processes described elsewhere herein.

FIG. 31 shows a system 3100 for isolating and/or purifying one or more target analytes from a urine sample. The system may include an epitachophoresis (ETP) device 3110. The ETP device may include a circular first electrode disposed at an outer edge of a circular channel. The circular channel may be any described herein. The ETP device may also include a sample collection reservoir in the center of the circular channel. The sample collection reservoir may be any sample collection reservoir or electrode reservoir described herein. The sample collection reservoir may be a cavity within the center of the circular channel. The sample collection reservoir may have a circular opening to the circular channel. The one or more target analytes in one or more focused zones may flow into the sample collection reservoir. The ETP device may further include a second electrode. The second electrode may be configured to be in closer electrical communication with the sample collection reservoir than the circular first electrode is with the sample collection reservoir. Closer electrical communication may refer to the resistance being lower or the current being higher given the same voltage applied. In addition, the ETP device may include a power supply configured to deliver a voltage difference between the circular first electrode and the second electrode.

System 3100 may include a sample concentrating device 3120 configured to increase the concentration of one or more target analytes within a sample at least 10 times higher. In some aspects, the sample concentrating device may include a centrifuge. In some embodiments, the centrifuge may be used for extracellular vesicle enrichment. In some aspects, the sample concentrating device comprises a vacuum filter. The sample concentrating device may include dialysis tubes, which may be configured to perform desalting and/or buffer exchange. The sample concentrating device may also include a heat block, which may be configured to reach a temperature for cell lysis and/or protein degradation. The sample concentrating device may be any device for concentrating the urine sample described herein.

System 3100 may include a robotic handler 3130. For example, robotic handler 3130 may be an automated robotic handling device configured to transfer an output from sample concentrating device 3120 to ETP device 3110. In addition, robotic handler 3130 may be configured to transfer an output from ETP device 3110 to a further analytical device, which may be any such device described herein.

System 3100 may include processor 3140. Processor 3140 may control ETP device 3110, sample concentrating device 3120, and/or robotic handler 3130 to perform any of the steps in process 3000. Processor 3140 may be part of a computer system.

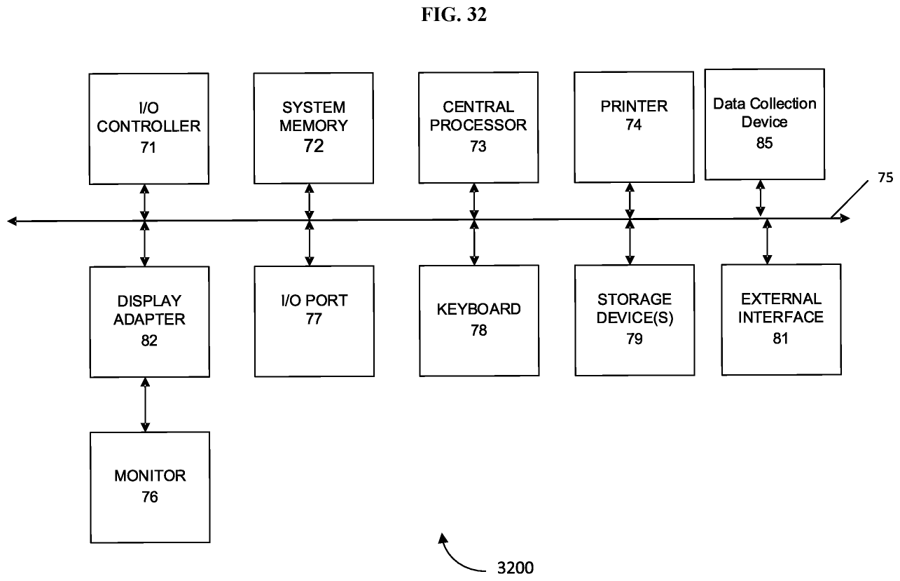

Any of the computer systems mentioned herein may utilize any suitable number of subsystems. Examples of such subsystems are shown in FIG. 32 in computer system 1200. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 32 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB). For example, I/O port 77 or external interface 81 (e.g. Ethernet, Wi-Fi, etc.) can be used to connect computer system 1200 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

In the preceding procedures, various steps have been described. It will, however, be evident that various modifications and changes may be made thereto, and additional procedures may be implemented, without departing from the broader scope of the procedures as set forth in the claims that follow.

1. A method of isolating and/or purifying one or more target analytes from a urine sample, the method comprising:
concentrating the urine sample to form a concentrated urine sample, the concentrated urine sample having a concentration of the one or more target analytes that is at least 10 times higher than an initial concentration of the one or more target analytes in the urine sample;
adding the concentrated urine sample to a first electrolyte to form a first mixture;
applying a voltage difference between a first electrode and a second electrode, wherein:
the first electrode is disposed in the first mixture,
the second electrode is disposed in a second electrolyte, and
the first electrolyte is different from the second electrolyte;
flowing, using the voltage difference, the one or more target analytes in one or more focused zones within the second electrolyte to the second electrode; and
collecting the one or more target analytes by collecting a second mixture comprising the one or more focused zones, wherein the concentration of any of the one or more target analytes in the second mixture is higher than the concentration of the respective target analyte in the concentrated urine sample.

2. The method of claim 1, wherein concentrating the urine sample comprises one or more of vacuum filtration, desalting, buffer exchange, extracellular vesicle enrichment, exosome enrichment, cell lysis, protein degradation, or centrifugation-based cell-removal.

3. The method of claim 2, wherein:
concentrating the urine sample comprises buffer exchange, and
the pH of the concentrated urine sample is in a range from 6.0 to 8.5.

4. The method of claim 1, wherein concentrating the urine sample comprises centrifuging the urine sample.

5. The method of claim 4, wherein centrifuging the urine sample comprises centrifuging at a force in a range from 1,000 to 3,600×g.

6. The method of claim 1, wherein the one or more target analytes comprise DNA, RNA, or a combination thereof.

7. The method of claim 1, wherein the one or more target analytes comprise cell-free nucleic acids, circulating tumor nucleic acids, biomarkers, proteins, extracellular vesicles, or a combination thereof.

8. The method of claim 1, wherein concentrating the urine sample comprises filtering the urine sample by molecular weight.

9. The method of claim 1, wherein filtering the urine sample by molecular weight comprises removing components of the urine sample with a molecular weight above or below a cutoff in a range of 3,000 to 10,000.

10. The method of claim 1, wherein the concentration of the one or more target analytes in the concentrated urine sample is at least 40 times higher than the initial concentration.

11. The method of claim 1, wherein the concentration of any of the one or more target analytes in the second mixture is at least two times higher than the concentration of the respective target analyte in the concentrated urine sample.

12. The method of claim 1, wherein the volume of the urine sample is from 10 ml to 50 ml.

13. The method of claim 1, wherein the volume of the concentrated urine sample is 0.5 ml to 2.5 ml.

14. The method of claim 1, wherein the second mixture comprises 2 or more focused zones.

15. The method of claim 1, further comprising desalting the urine sample, wherein the concentrated urine sample is desalted.

16. The method of claim 1, further comprising sequencing a target analyte of the one or more target analytes in the second mixture.

17. A system comprising:
an epitachophoresis device, the epitachophoresis device comprising:
a circular first electrode disposed at an outer edge of a circular channel,
a sample collection reservoir in the center of the circular channel,
a second electrode, the second electrode configured to be in closer electrical communication with the sample collection reservoir than the circular first electrode is with the sample collection reservoir, and
a power supply configured to deliver a voltage difference between the circular first electrode and the second electrode; and
a sample concentrating device configured to increase the concentration of one or more target analytes within a sample at least 10 times higher.

18. The system of claim 17, wherein the sample concentrating device comprises a centrifuge.

19. The system of claim 17, wherein the sample concentrating device comprises a vacuum filter.

20. The system of claim 17, further comprising an automated robotic handling device configured to transfer an output from the sample concentrating device to the epitachophoresis device.

21. A method of isolating and/or purifying one or more target analytes from a urine or other high salt comprising biological sample, such as sodium or potassium salts, which sample potentially comprises one or more target analytes, wherein said method comprises:
a. providing a device for effecting epitachophoresis (“ETP”);
b. providing the sample comprising said one or more target analytes;
c. performing one or more epitachophoresis runs by effecting ETP using said device to focus said one or more target analytes into one or more focused zones;
d. collecting said one or more target analytes by collecting said one or more focused zones comprising said one or more target analytes; and
thereby obtaining one or more isolated and/or purified target analytes, optionally wherein said target analytes comprise one or more nucleic acids.

22. The method of claim 21, wherein prior to step c., a sample solution is prepared by effecting one or more sample pretreatment steps on said urine or other high salt comprising biological sample.

23. The method of claim 21, wherein the sample comprises a urine sample.

24. The method of claim 22, wherein the one or more sample pretreatment steps comprise one or more of vacuum filtration, desalting, buffer exchange, extracellular vesicle enrichment, exosome enrichment, cell lysis, protein degradation, centrifugation-based cell-removal, and/or concentration steps.

25. The method of claim 24, wherein said desalting, buffer exchange, extracellular vesicle enrichment, exosome enrichment step, or concentration steps are centrifugation-based.

26. The method of claim 1, wherein the one or more target analytes comprise any one or more of one or more nucleic acids; one or more proteins; one or more cells; one or more extracellular vesicles; one or more one or more exosomes, microvesicles, and/or apoptotic bodies, optionally one or more urinary exosomes; and/or one or more biomarkers.

27. The method of claim 1, wherein the one or more target analytes comprise DNA or RNA.

28. The method of claim 1, wherein the one or more target analytes comprise one or more circulating nucleic acids.

29. The method of claim 1, wherein the isolated or purified target analytes comprise DNA or RNA.

30. The method of claim 1, wherein the quantity of nucleic acids isolated and/or purified is greater as compared to the quantity of nucleic acids obtained using a column-based or bead-based protocol as measured by a fluorometer-based method.
